SEQUENCE LISTING (30 sequences)

SEQ ID NO: 1 - DNA, 1320 bp, Synechocystis sp. PCC 6803
gtgtgttgttggcaatcgagaggtctgcttgtgaaacgtgtcttagcgattatcctgggcggtggggccgggacccgcctctatcctttaaccaaactcagagccaaacccgcagttcccttggccggaaagtatcgcctcatcgatattcccgtcagtaattgcatcaactcagaaatcgttaaaatttacgtccttacccagtttaattccgcctcccttaaccgtcacatcagccgggcctataatttttccggcttccaagaaggatttgtggaagtcctcgccgcccaacaaaccaaagataatcctgattggtttcagggcactgctgatgcggtacggcaatacctctggttgtttagggaatgggacgtagatgaatatcttattctgtccggcgaccatctctaccgcatggattacgcccaatttgttaaaagacaccgggaaaccaatgccgacataaccctttccgttgtgcccgtggatgacagaaaggcacccgagctgggcttaatgaaaatcgacgcccagggcagaattactgacttttctgaaaagccccagggggaagccctccgggccatgcaggtggacaccagcgttttgggcctaagtgcggagaaggctaagcttaatccttacattgcctccatgggcatttacgttttcaagaaggaagtattgcacaacctcctggaaaaatatgaaggggcaacggactttggcaaagaaatcattcctgattcagccagtgatcacaatctgcaagcctatctctttgatgactattgggaagacattggtaccattgaagccttctatgaggctaatttagccctgaccaaacaacctagtcccgactttagtttttataacgaaaaagcccccatctataccaggggtcgttatcttccccccaccaaaatgttgaattccaccgtgacggaatccatgatcggggaaggttgcatgattaagcaatgtcgcatccaccactcagttttaggcattcgcagtcgcattgaatctgattgcaccattgaggatactttggtgatgggcaatgatttctacgaatcttcatcagaacgagacaccctcaaagcccggggggaaattgccgctggcataggttccggcaccactatccgccgagccatcatcgacaaaaatgcccgcatcggcaaaaacgtcatgattgtcaacaaggaaaatgtccaggaggctaaccgggaagagttaggtttttacatccgcaatggcatcgtagtagtgattaaaaatgtcacgatcgccgacggcacggtaatctag

SEQ ID NO: 2 - DNA, 1293 bp, Synechococcus elongatus PCC 7942
gtgaaaaacgtgctggcgatcattctcggtggaggcgcaggcagtcgtctctatccactaaccaaacagcgcgccaaaccagcggtccccctggcgggcaaataccgcttgatcgatattcccgtcagcaattgcatcaacgctgacatcaacaaaatctatgtgctgacgcagtttaactctgcctcgctcaaccgccacctcagtcagacctacaacctctccagcggctttggcaatggctttgttgaggtgctagcagctcagattacgccggagaaccccaactggttccaaggcaccgccgatgcggttcgccagtatctctggctaatcaaagagtgggatgtggatgagtacctgatcctgtcgggggatcatctctaccgcatggactatagccagttcattcagcggcaccgagacaccaatgccgacatcacactctcggtcttgccgatcgatgaaaagcgcgcctctgattttggcctgatgaagctagatggcagcggccgggtggtcgagttcagcgaaaagcccaaaggggatgaactcagggcgatgcaagtcgataccacgatcctcgggcttgaccctgtcgctgctgctgcccagcccttcattgcctcgatgggcatctacgtcttcaagcgggatgttctgatcgatttgctcagccatcatcccgagcaaaccgactttggcaaggaagtgattcccgctgcagccacccgctacaacacccaagcctttctgttcaacgactactgggaagacatcggcacgatcgcctcattctacgaggccaatctggcgctgactcagcaacctagcccacccttcagcttctacgacgagcaggcgccgatttacacccgcgctcgctacctgccgccaaccaagctgctcgattgccaggtgacccagtcgatcattggcgagggctgcattctcaagcaatgcaccgttcagaattccgtcttagggattcgctcccgcattgaggccgactgcgtgatccaggacgccttgttgatgggcgctgacttctacgaaacctcggagctacggcaccagaatcgggccaatggcaaagtgccgatgggaatcggcagtggcagcaccatccgtcgcgccatcgtcgacaaaaatgcccacattggccagaacgttcagatcgtcaacaaagaccatgtggaagaggccgatcgcgaagatctgggctttatgatccgcagcggcattgtcgttgtggtcaaaggggcggttattcccgacaacacggtgatctaa

SEQ ID NO: 3 - DNA, 1296 bp, Synechococcus sp. WH8102
atgaagcgggttttggccatcattctcggcggcggtgccgggactcgtctctacccgctcaccaagatgcgcgccaagccggccgtccccttggccggtaagtatcgactgattgatatccccatcagcaactgcatcaactcgaacatcaacaagatgtacgtgatgacgcagttcaacagtgcgtctctcaatcgtcacctcagccagacgttcaacctgagcgcatccttcggtcagggattcgtcgaggtgcttgctgcccagcagacgcctgacagtccatcctggtttgaaggcactgccgacgctgtgcggaagtaccagtggctgttccaggaatgggatgtcgatgaatacctgatcctgtccggtgaccagctgtaccggatggattacagcctgttcgttgaacatcaccgcagcactggtgctgacctcaccgttgcagcccttcctgtggacccgaaacaggccgaggcgttcggcttgatgcgcacggatggtgacggagacatcaaggagttccgcgaaaagcccaagggtgattctttgcttgagatggcggttgacaccagccgatttggactcagtgcgaattcggccaaggagcgtccctacctggcgtcgatggggatttatgtcttcagcagagacactctgttcgacctgctcgattccaatcctggttataaggacttcggcaaggaagtcattcctgaggccctcaagcgtggcgacaagctgaagagctatgtctttgacgattattgggaagatatcggaacgatcggagcgttctacgaggccaacctggcgctcacccagcaacccacaccccccttcagcttctacgacgagaagttcccgatctacactcgtccccgctatttacccccgagcaaactggttgatgctcagatcaccaattcgatcgttggcgaaggctcaattttgaagtcatgcagcattcatcactgcgttttgggtgttcgcagtcgcattgaaaccgatgtggtgctgcaagacaccttggtgatgggcgctgacttctttgaatccagtgatgagcgtgccgtgcttcgcgagcgtggtggtattccggtcggggtgggccaaggtacgactgtgaagcgcgccatcctcgataaaaacgctcgcatcggatccaacgtcaccatcgtcaacaaggatcacgtcgaggaagctgatcgttccgatcagggcttctatattcgtaatggcattgttgttgttgtcaagaacgccaccatccaggacggaactgtgatctga

SEQ ID NO: 4 - DNA, 1296 bp, Synechococcus sp. RCC 307
atgaaacgggttctcgcaatcattctcggtggcggtgcgggtacgcggctctatccgctgaccaaaatgcgggccaaaccagccgtgccgctggcgggtaagtaccgcctcatcgacatccccgttagcaactgcatcaacagcgggatcaacaagatctatgtgctgacgcagttcaacagcgcatcactgaatcgccacatcgctcaaaccttcaacctctcctcggggtttgatcaagggtttgttgaagttctggcggcccagcagaccccagatagccccagttggtttgaaggaacagccgatgctgttcgtaaatacgaatggctgctgcaggagtgggacatcgacgaagtgctgatcctttcgggtgaccagctctaccggatggactatgcccattttgtggctcagcaccgcgccagcggcgctgacctcaccgtggccgccctcccggttgatcgcgagcaagcccagagctttggcttgatgcacaccggtgcagaagcctccatcaccaagttccgcgaaaagcccaaaggcgaggcactcgatgagatgtcctgcgataccgccagcatgggcttgagcgctgaggaagcccatcgccggccgttcctggcttccatgggcatctacgtgttcaagcgggacgtgctcttccgcttactggctgaaaaccccggtgccactgacttcggtaaggagatcatccccaaggcactcgacgatggcttcaaactccgctcctatctcttcgacgattactgggaagacatcggaaccatccgtgctttctatgaagcgaatctggcgctgacgacccagccgcgtccgcccttctctttctacgacaagcgtttcccgatctacacacgtcatcgctacctgccgccctccaagcttcaagatgcgcaggtcaccgactccattgttggtgaggggtccattttgaaggcttgcagtattcaccactgcgtcttgggtgtgcgcagccgcattgaagacgaggttgccttgcaagacaccctggtgatgggcaacgacttctatgagtccggcgaagagcgggccatcctgcgggaacgtggtggcatccccatgggtgtgggccgaggaaccacggtgaaaaaggccatcctcgataagaacgtccgcatcggcagcaacgtcagcatcatcaacaaagacaacgttgaggaagccgaccgcgctgagcagggcttctacatccgtggcgggattgtggtgatcaccaaaaacgcttcgattcccgacgggatggtgatctga

SEQ ID NO: 5 - DNA, 1290 bp, Synechococcus sp. PCC 7002
gtgaaacgagtcctaggaatcatacttggcggcggcgcaggtactcgcctatatccgctaacaaaactcagagctaagcccgcagtacctctagcaggcaaatatcgtctcattgatattcctgttagcaattgcattaattctgaaattcataaaatctacattttaacccaatttaattcagcatctttaaatcgtcacattagtcgaacctacaactttaccggcttcaccgaaggctttaccgaagtactcgcagcccaacaaactaaagaaaatcccgattggttccaaggcaccgccgacgctgtccgacagtacagttggcttctagaagactgggatgtcgatgaatacatcattctctccggtgatcacctctaccgtatggattaccgtgaatttatccagcgccaccgtgacactggggcagacatcaccctgtctgtggttcccgtgggcgaaaaagtagcccccgcctttgggttgatgaaaattgatgccaatggtcgtgtcgtggactttagtgaaaagcccactggtgaagcccttaaggcgatgcaggtggatacccagtccttgggtctcgatccagagcaggcgaaagaaaagccctacattgcgtcgatggggatctacgtctttaagaaacaagtactcctcgatctactcaaagaaggcaaagataaaaccgatttcgggaaagaaattattcctgatgcggccaaggactacaacgttcaggcctatctctttgatgattattgggctgacattgggaccatcgaagcgttctatgaagcaaaccttggcttgacgaagcagccgatcccaccctttagtttctatgacgaaaaggctcccatctacacccgggcgcgctacttaccgccgacgaaggtgctcaacgctgacgtgacagaatcgatgatcagcgaaggttgcatcattaaaaactgccgcattcaccactcagttcttggcattcgcacccgtgtcgaagcggactgcactatcgaagatacgatgatcatgggcgcagattattatcagccctatgagaagcgccaggattgtctccgtcgtggcaagcctcccattgggattggtgaagggacaacgattcgccgggcgatcatcgataaaaatgcacgcatcggtaaaaacgtgatgatcgtcaataaggaaaatgtggaggagtcaaaccgtgaggagcttggctactacattcgcagcggcattacagtggtgctaaagaacgccgttattcccgacggtacggtcatttaa

SEQ ID NO: 6 - protein, 439 aa, Synechocystis sp. PCC 6803
MCCWQSRGLLVKRVLAIILGGGAGTRLYPLTKLRAKPAVPLAGKYRLIDIPVSNCINSEIVKIYVLTQFNSASLNRHISRAYNFSGFQEGFVEVLAAQQTKDNPDWFQGTADAVRQYLWLFREWDVDEYLILSGDHLYRMDYAQFVKRHRETNADITLSVVPVDDRKAPELGLMKIDAQGRITDFSEKPQGEALRAMQVDTSVLGLSAEKAKLNPYIASMGIYVFKKEVLHNLLEKYEGATDFGKEIIPDSASDHNLQAYLFDDYWEDIGTIEAFYEANLALTKQPSPDFSFYNEKAPIYTRGRYLPPTKMLNSTVTESMIGEGCMIKQCRIHHSVLGIRSRIESDCTIEDTLVMGNDFYESSSERDTLKARGEIAAGIGSGTTIRRAIIDKNARIGKNVMIVNKENVQEANREELGFYIRNGIVVVIKNVTIADGTVI

SEQ ID NO: 7 - protein, 430 aa, Synechococcus elongatus PCC 7942
MKNVLAIILGGGAGSRLYPLTKQRAKPAVPLAGKYRLIDIPVSNCINADINKIYVLTQFNSASLNRHLSQTYNLSSGFGNGFVEVLAAQITPENPNWFQGTADAVRQYLWLIKEWDVDEYLILSGDHLYRMDYSQFIQRHRDTNADITLSVLPIDEKRASDFGLMKLDGSGRVVEFSEKPKGDELRAMQVDTTILGLDPVAAAAQPFIASMGIYVFKRDVLIDLLSHHPEQTDFGKEVIPAAATRYNTQAFLFNDYWEDIGTIASFYEANLALTQQPSPPFSFYDEQAPIYTRARYLPPTKLLDCQVTQSIIGEGCILKQCTVQNSVLGIRSRIEADCVIQDALLMGADFYETSELRHQNRANGKVPMGIGSGSTIRRAIVDKNAHIGQNVQIVNKDHVEEADREDLGFMIRSGIVVVVKGAVIPDNTVI

SEQ ID NO: 8 - protein, 431 aa, Synechococcus sp. WH8102
MKRVLAIILGGGAGTRLYPLTKMRAKPAVPLAGKYRLIDIPISNCINSNINKMYVMTQFNSASLNRHLSQTFNLSASFGQGFVEVLAAQQTPDSPSWFEGTADAVRKYQWLFQEWDVDEYLILSGDQLYRMDYSLFVEHHRSTGADLTVAALPVDPKQAEAFGLMRTDGDGDIKEFREKPKGDSLLEMAVDTSRFGLSANSAKERPYLASMGIYVFSRDTLFDLLDSNPGYKDFGKEVIPEALKRGDKLKSYVFDDYWEDIGTIGAFYEANLALTQQPTPPFSFYDEKFPIYTRPRYLPPSKLVDAQITNSIVGEGSILKSCSIHHCVLGVRSRIETDVVLQDTLVMGADFFESSDERAVLRERGGIPVGVGQGTTVKRAILDKNARIGSNVTIVNKDHVEEADRSDQGFYIRNGIVVVVKNATIQDGTVI

SEQ ID NO: 9 - protein, 431 aa, Synechococcus sp. RCC 307
MKRVLAIILGGGAGTRLYPLTKMRAKPAVPLAGKYRLIDIPVSNCINSGINKIYVLTQFNSASLNRHIAQTFNLSSGFDQGFVEVLAAQQTPDSPSWFEGTADAVRKYEWLLQEWDIDEVLILSGDQLYRMDYAHFVAQHRASGADLTVAALPVDREQAQSFGLMHTGAEASITKFREKPKGEALDEMSCDTASMGLSAEEAHRRPFLASMGIYVFKRDVLFRLLAENPGATDFGKEIIPKALDDGFKLRSYLFDDYWEDIGTIRAFYEANLALTTQPRPPFSFYDKRFPIYTRHRYLPPSKLQDAQVTDSIVGEGSILKACSIHHCVLGVRSRIEDEVALQDTLVMGNDFYESGEERAILRERGGIPMGVGRGTTVKKAILDKNVRIGSNVSIINKDNVEEADRAEQGFYIRGGIVVITKNASIPDGMVI

SEQ ID NO: 10 - protein, 429 aa, Synechococcus sp. PCC 7002
MKRVLGIILGGGAGTRLYPLTKLRAKPAVPLAGKYRLIDIPVSNCINSEIHKIYILTQFNSASLNRHISRTYNFTGFTEGFTEVLAAQQTKENPDWFQGTADAVRQYSWLLEDWDVDEYIILSGDHLYRMDYREFIQRHRDTGADITLSVVPVGEKVAPAFGLMKIDANGRVVDFSEKPTGEALKAMQVDTQSLGLDPEQAKEKPYIASMGIYVFKKQVLLDLLKEGKDKTDFGKEIIPDAAKDYNVQAYLFDDYWADIGTIEAFYEANLGLTKQPIPPFSFYDEKAPIYTRARYLPPTKVLNADVTESMISEGCIIKNCRIHHSVLGIRTRVEADCTIEDTMIMGADYYQPYEKRQDCLRRGKPPIGIGEGTTIRRAIIDKNARIGKNVMIVNKENVEESNREELGYYIRSGITVVLKNAVIPDGTVI

SEQ ID NO: 11 - DNA, 1434 bp, Synechocystis sp. PCC 6803
atgaagattttatttgtggcggcggaagtatcccccctagcaaaggtaggtggcatgggggatgtggtgggttccctgcctaaagttctgcatcagttgggccatgatgtccgtgtcttcatgccctactacggtttcatcggcgacaagattgatgtgcccaaggagccggtctggaaaggggaagccatgttccagcagtttgctgtttaccagtcctatctaccggacaccaaaattcctctctacttgttcggccatccagctttcgactcccgaaggatctatggcggagatgacgaggcgtggcggttcacttttttttctaacggggcagctgaatttgcctggaaccattggaagccggaaattatccattgccatgattggcacactggcatgatccctgtttggatgcatcagtccccagacatcgccaccgttttcaccatccataatcttgcttaccaagggccctggcggggcttgcttgaaactatgacttggtgtccttggtacatgcagggagacaatgtgatggcggcggcgattcaatttgccaatcgggtgactaccgtttctcccacctatgcccaacagatccaaaccccggcctatggggaaaagctggaagggttattgtcctacctgagtggtaatttagtcggtattctcaacggtattgatacggagatttacaacccggcggaagaccgctttatcagcaatgttttcgatgcggacagtttggacaagcgggtgaaaaataaaattgccatccaggaggaaacggggttagaaattaatcgtaatgccatggtggtgggtatagtggctcgcttggtggaacaaaaggggattgatttggtgattcagatccttgaccgcttcatgtcctacaccgattcccagttaattatcctcggcactggcgatcgccattacgaaacccaactttggcagatggcttcccgatttcctgggcggatggcggtgcaattactccacaacgatgccctttcccgtcgagtctatgccggggcggatgtgtttttaatgccttctcgctttgagccctgtgggctgagtcaattgatggccatgcgttatggctgtatccccattgtgcggcggacagggggtttggtggatacggtatccttctacgatcctatcaatgaagccggcaccggctattgctttgaccgttatgaacccctggattgctttacggccatggtgcgggcctgggagggtttccgtttcaaggcagattggcaaaaattacagcaacgggccatgcgggcagactttagttggtaccgttccgccggggaatatatcaaagtttataagggcgtggtggggaaaccggaggaattaagccccatggaagaggaaaaaatcgctgagttaactgcttcctatcgctaa

SEQ ID NO: 12 - DNA, 1398 bp, Synechococcus elongatus PCC 7942
atgcggattctgttcgtggctgccgaatgtgctcccttcgccaaagtgggaggcatgggagatgtggttggttccctgcccaaagtgctgaaagctctgggccatgatgtccgaatcttcatgccgtactacggctttctgaacagtaagctcgatattcccgctgaaccgatctggtggggctacgcgatgtttaatcacttcgcggtttacgaaacgcagctgcccggttcagatgtgccgctctacttaatggggcatccagcttttgatccgcatcgcatctactcaggagaagacgaagactggcgcttcacgttttttgccaatggggctgctgaattttcttggaactactggaaaccacaagtcattcactgccacgattggcacactgggatgattccggtttggatgcaccagtccccggatatctcgactgtcttcaccattcataacttggcctaccaagggccgtggcgctggaagctcgagaaaatcacctggtgcccttggtacatgcagggcgacagcaccatggcggcggccttgctctatgccgatcgcgtcaacacggtatcgcccacctatgcccagcagattcaaacaccgacctacggtgaaaagctggagggtcttctctcatttatcagtggcaagctaagcggcatccttaacgggattgatgttgatagctacaaccctgcaacggatacgcggattgtggccaactacgatcgcgacactcttgataaacgactgaacaataagctggcgctccaaaaggagatggggcttgaggtcaatcccgatcgcttcctgattggctttgtggctcgtctagtcgagcagaagggcattgacttgctgctgcaaattcttgatcgctttctgtcttacagcgatgcccaatttgttgtcttaggaacgggcgagcgctactacgaaacccagctctgggagttggcgacccgctatccgggccggatgtccacttatctgatgtacgacgaggggctgtcgcgacgcatttatgccggtagcgacgccttcttggtgccctctcgttttgaaccttgcggtatcacgcaaatgctggcactgcgctacggcagtgtgccgattgtgcgccgtacgggggggttggtcgatacggtcttccaccacgatccgcgtcatgccgagggcaatggctattgcttcgatcgctacgagccgctggacctctatacctgtctggtgcgggcttgggagagttaccagtaccagccccaatggcaaaagctacagcaacggggtatggccgttgatctgagctggaaacaatcggcgatcgcctacgaacagctctacgctgaagcgattgggctaccgatcgatgtcttacaggaggcctag

SEQ ID NO: 13 - DNA, 1542 bp, Synechococcus sp. WH8102
atgcgcatcctcttcgctgccgcggaatgcgccccgatgatcaaggtcggtggcatgggggatgtggtgggatcgctgcctccggctctggccaagcttggccacgacgtgcggctgatcatgccgggctactccaagctctggaccaagctgacgatctcggacgaacccatctggcgcgcccagacgatgggtacggaattcgcggtttacgagacgaagcatccaggcaatgggatgaccatctacctggtgggacatccggtgttcgatcccgagcggatctatggcggtgaagatgaggactggcgcttcaccttctttgccagtgccgccgctgaattcgcctggaatgtctggaagccgaatgttcttcactgccacgactggcacaccggcatgattccggtctggatgcaccaggacccggagatcagcacggtcttcaccatccacaacctcaagtaccagggcccctggcgttggaagctggatcgcatcacctggtgcccctggtacatgcagggagatcacaccatggcggcggcacttctgtacgccgaccgggtcaacgccgtctcccccacctacgccgaggaaatccgtacggcggagtacggcgaaaagctggatggtttgctcaatttcgtctccggcaagctgcgcggcatcctcaatggcattgacctcgaggcctggaacccccagaccgatggggctctgccggccaccttcagcgccgacgacctctccggtaaagcggtctgcaagcgggtgttgcaggagcgcatgggtcttgaggtgcgtgacgacgcctttgtcctcggcatggtcagccgactcgtcgatcagaagggcgtcgatctgcttctgcaggtggcggaccgtttgctcgcctacaccgacacgcagatcgtggtgctcggcaccggtgaccgtggcctggaatccggcctgtggcagctggcctcccgccatgccggccgttgcgccgtcttcctcacctacgacgacgacctctcccgactgatctatgccggcagtgacgccttcctgatgcccagtcgcttcgagccctgcggcatcagccagctgtacgccatgcgttacggctccgttcctgtggtgcgcaaggtgggcggcctggtggacaccgttcctccccacagtccagctgatgccagcgggaccggcttctgcttcgatcgttttgagccggtcgacttctacaccgcattggtgcgtgcctgggaggcctaccgccatcgcgacagctggcaggagttgcagaagcgcggcatgcagcaggactacagctgggaccgttcggccatcgattacgacgtcatgtaccgcgatgtctgcggtctgaaggaacccacccctgatgccgcgatggtggaacagttctcccagggacaggctgcggatccctcccgcccagaggatgatgcgatcaatgctgctcccgaggcggtcaccgcgccgtccggccccagccgcaacccccttaatcgtctcttcggccgcagggccgactga

SEQ ID NO: 14 - DNA, 1524 bp, Synechococcus sp RCC 307
atgcgcatcctctttgctgcggccgaatgcgcaccgatggtgaaagtcggcggcatgggagatgtggtgggatctctgcctccagccctcgctgagttgggtcacgacgtgcgcgtgatcatgcccggctacggcaagctctggtcccagcttgatgtgcccagcgagccgatctggcgtgcccaaaccatgggcaccgattttgctgtctatgagacccgtcaccccaagaccgggctcacgatctatttggtgggccatccggtttttgatggtgagcgcatctatggaggtgaagacgaggactggcgcttcaccttcttcgctagcgccacctccgaatttgcctggaacgcttggaagccccaggtgctgcattgccatgactggcacaccggcatgattccggtgtggatgcaccaagaccccgagatcagcacggtcttcaccatccacaacctcaaatatcaaggtccctggcgctggaagctcgagcgcatgacctggtgcccctggtacatgcagggcgaccacaccatggcggcagccttgctgtatgccgaccgcgtcaatgcggtttcacccacctacgcccaagagatccgcacgccggaatacggcgaacaactggaggggttgctgaactacatcagcggcaagctgcgaggcatcctcaatggcatcgatgtggaggcttggaatcccgccactgattcgcggattccggccacctacagcactgctgacctcagtggcaaagccgtctgcaagcgggctctgcaagagcgcatggggcttcaggtgaaccccgacacctttgtgatcggtttggtgagccgtttggtggaccaaaaaggcgtcgacctgctgctgcaggttgccgaacgcttccttgcctacaccgatacgcagatcgttgtgttgggcaccggggatcgccatttggaatcgggcctgtggcaaatggcgagtcagcacagcggccgcttcgcttccttcctcacctacgacgatgatctctcccggctgatctacgccggcagtgatgccttcttgatgccctcgcgctttgagccctgcggcatcagccagttgctctcgatgcgctacggcaccatcccggtggtgcgccgcgtcggtggactggtcgacaccgtgcctccctatgttcccgccacccaagagggcaatggcttctgcttcgaccgctatgaagcgatcgacctttacaccgccttggtgcgcgcctgggaggcctaccgccatcaagacagctggcagcaattgatgaagcgggtgatgcaggttgatttcagctgggctcgttccgccttggaatacgaccgcatgtatcgcgatgtttgcggaatgaaggagcccacgccggaagccgatgcggtggcggccttctccattccccagccgcctgaacagcaggccgcacgtgctgccgctgaagccgctgaccccaacccccaacggcgctttaatccccttggattgctgcgccgaaacggcggttga

SEQ ID NO: 15 - DNA, 1437 bp, Synechococcus sp. PCC 7002
atgcgtattttgtttgtttctgccgaggctgctcccatcgctaaagctggaggcatgggagatgtggtgggatcactgcctaaagttttacggcagttaggacatgacgcgagaattttcttaccctattacggctttctcaacgacaaactcgacatccctgcagaacccgtttggtggggcagtgcgatgttcaatacttttgccgtttatgaaactgtgttgcccaacaccgatgtccccctttatctgtttggccatcccgcctttgatggacggcatatttatggtgggcaggatgaattttggcgctttaccttttttgccaatggggccgctgaatttatgtggaaccactggaaaccccagatcgcccactgtcacgactggcacacgggcatgattccggtatggatgcaccaatcgccggatatcagtacggtgtttacgatccacaacttagcctaccaagggccttggcggggtttcctggagcgcaatacttggtgtccctggtatatggatggtgataacgtgatggcttcggcgctgatgtttgccgatcaggtgaacaccgtatctcccacctatgcccaacaaatccaaaccaaagtctatggtgaaaaattagagggtttgttgtcttggatcagtggcaaaagtcgcggcatcgtgaatggtattgacgtagaactttataatccttctaacgatcaagccctggtgaagcaattttctacgactaatcttgaggatcgggccgccaacaaagtgattatccaagaagaaacggggctagaggtcaactccaaggcttttttgatggcgatggtcacccgcttagtggaacaaaagggcattgatctgctgctaaatatcctggagcagtttatggcatacactgacgcccagctcattatcctcggcactggcgatcgccactacgaaacccaactctggcagactgcctaccgctttaaggggcggatgtccgtgcaactgctctataatgatgccctctcccgccggatttacgctggatccgatgtctttttgatgccgtcacgctttgagccctgtggcattagtcaaatgatggcgatgcgctacggttctgtaccgattgtgcggcgcaccgggggtttggtggatacggtctctttccatgatccgattcaccaaaccgggacaggctttagttttgaccgctacgaaccgctggatatgtacacctgcatggtgcgggcttgggaaagtttccgctacaaaaaagactgggctgaactacaaagacgaggcatgagccatgactttagttggtacaaatctgccggggaatatctcaagatgtaccgccaaagcattaaagaagctccggaattaacgaccgatgaagccgaaaaaatcacctatttagtgaaaaaacacgccatttaa

SEQ ID NO: 16 - protein, 477 aa, Synechocystis sp. PCC 6803
MKILFVAAEVSPLAKVGGMGDVVGSLPKVLHQLGHDVRVFMPYYGFIGDKIDVPKEPVWKGEAMFQQFAVYQSYLPDTKIPLYLFGHPAFDSRRIYGGDDEAWRFTFFSNGAAEFAWNHWKPEIIHCHDWHTGMIPVWMHQSPDIATVFTIHNLAYQGPWRGLLETMTWCPWYMQGDNVMAAAIQFANRVTTVSPTYAQQIQTPAYGEKLEGLLSYLSGNLVGILNGIDTEIYNPAEDRFISNVFDADSLDKRVKNKIAIQEETGLEINRNAMVVGIVARLVEQKGIDLVIQILDRFMSYTDSQLIILGTGDRHYETQLWQMASRFPGRMAVQLLHNDALSRRVYAGADVFLMPSRFEPCGLSQLMAMRYGCIPIVRRTGGLVDTVSFYDPINEAGTGYCFDRYEPLDCFTAMVRAWEGFRFKADWQKLQQRAMRADFSWYRSAGEYIKVYKGVVGKPEELSPMEEEKIAELTASYR

SEQ ID NO: 17 - protein, 465 aa, Synechococcus elongatus PCC 7942
MRILFVAAECAPFAKVGGMGDVVGSLPKVLKALGHDVRIFMPYYGFLNSKLDIPAEPIWWGYAMFNHFAVYETQLPGSDVPLYLMGHPAFDPHRIYSGEDEDWRFTFFANGAAEFSWNYWKPQVIHCHDWHTGMIPVWMHQSPDISTVFTIHNLAYQGPWRWKLEKITWCPWYMQGDSTMAAALLYADRVNTVSPTYAQQIQTPTYGEKLEGLLSFISGKLSGILNGIDVDSYNPATDTRIVANYDRDTLDKRLNNKLALQKEMGLEVNPDRFLIGFVARLVEQKGIDLLLQILDRFLSYSDAQFVVLGTGERYYETQLWELATRYPGRMSTYLMYDEGLSRRIYAGSDAFLVPSRFEPCGITQMLALRYGSVPIVRRTGGLVDTVFHHDPRHAEGNGYCFDRYEPLDLYTCLVRAWESYQYQPQWQKLQQRGMAVDLSWKQSAIAYEQLYAEAIGLPIDVLQEA

SEQ ID NO: 18 - protein, 513 aa, Synechococcus sp. WH8102
MRILFAAAECAPMIKVGGMGDVVGSLPPALAKLGHDVRLIMPGYSKLWTKLTISDEPIWRAQTMGTEFAVYETKHPGNGMTIYLVGHPVFDPERIYGGEDEDWRFTFFASAAAEFAWNVWKPNVLHCHDWHTGMIPVWMHQDPEISTVFTIHNLKYQGPWRWKLDRITWCPWYMQGDHTMAAALLYADRVNAVSPTYAEEIRTAEYGEKLDGLLNFVSGKLRGILNGIDLEAWNPQTDGALPATFSADDLSGKAVCKRVLQERMGLEVRDDAFVLGMVSRLVDQKGVDLLLQVADRLLAYTDTQIVVLGTGDRGLESGLWQLASRHAGRCAVFLTYDDDLSRLIYAGSDAFLMPSRFEPCGISQLYAMRYGSVPVVRKVGGLVDTVPPHSPADASGTGFCFDRFEPVDFYTALVRAWEAYRHRDSWQELQKRGMQQDYSWDRSAIDYDVMYRDVCGLKEPTPDAAMVEQFSQGQAADPSRPEDDAINAAPEAVTAPSGPSRNPLNRLFGRRAD

SEQ ID NO: 19 - protein, 507 aa, Synechococcus sp RCC 307
MRILFAAAECAPMVKVGGMGDVVGSLPPALAELGHDVRVIMPGYGKLWSQLDVPSEPIWRAQTMGTDFAVYETRHPKTGLTIYLVGHPVFDGERIYGGEDEDWRFTFFASATSEFAWNAWKPQVLHCHDWHTGMIPVWMHQDPEISTVFTIHNLKYQGPWRWKLERMTWCPWYMQGDHTMAAALLYADRVNAVSPTYAQEIRTPEYGEQLEGLLNYISGKLRGILNGIDVEAWNPATDSRIPATYSTADLSGKAVCKRALQERMGLQVNPDTFVIGLVSRLVDQKGVDLLLQVAERFLAYTDTQIVVLGTGDRHLESGLWQMASQHSGRFASFLTYDDDLSRLIYAGSDAFLMPSRFEPCGISQLLSMRYGTIPVVRRVGGLVDTVPPYVPATQEGNGFCFDRYEAIDLYTALVRAWEAYRHQDSWQQLMKRVMQVDFSWARSALEYDRMYRDVCGMKEPTPEADAVAAFSIPQPPEQQAARAAAEAADPNPQRRFNPLGLLRRNGG

SEQ ID NO: 20 - protein, 478 aa, Synechococcus sp. PCC 7002
MRILFVSAEAAPIAKAGGMGDVVGSLPKVLRQLGHDARIFLPYYGFLNDKLDIPAEPVWWGSAMFNTFAVYETVLPNTDVPLYLFGHPAFDGRHIYGGQDEFWRFTFFANGAAEFMWNHWKPQIAHCHDWHTGMIPVWMHQSPDISTVFTIHNLAYQGPWRGFLERNTWCPWYMDGDNVMASALMFADQVNTVSPTYAQQIQTKVYGEKLEGLLSWISGKSRGIVNGIDVELYNPSNDQALVKQFSTTNLEDRAANKVIIQEETGLEVNSKAFLMAMVTRLVEQKGIDLLLNILEQFMAYTDAQLIILGTGDRHYETQLWQTAYRFKGRMSVQLLYNDALSRRIYAGSDVFLMPSRFEPCGISQMMAMRYGSVPIVRRTGGLVDTVSFHDPIHQTGTGFSFDRYEPLDMYTCMVRAWESFRYKKDWAELQRRGMSHDFSWYKSAGEYLKMYRQSIKEAPELTTDEAEKITYLVKKHAI

SEQ ID NO: 21 - DNA, 996 bp, Synechococcus elongatus PCC 7942
atgtcagaggctcaaacgcccctgacagtaccgaagaagtttcttggtgctccaggaggcttcaaccccaccgtcgcactcttcttggcaggttatacctgcgcggcgctctcagttttggggtactggtgctggagttggccccactggctatctttccttctgagtgtcacagccttacatttggtaggcaccgtcattcacgatgcctctcataatgtggctcacgccagtcgcattctgaatgcgattttgggacatggcagtgcactattgctgggctttacttttccggtgtttacgcgggttcacctgcaacatcacgcccacgtcaacgatcccaagaacgatcccgaccacatcgtttccacctttgggccgctgtggttgatcgcaccgcgcttcttctatcacgagatctatttcttccagcgccgcctttggaagaaatttgaattactcgaatggttcctcagtcgcgctgtggtcatcggcatctttgcctgcggcgtcaagtttggcttcctgggcttcctgatgaactactggctggctccagccttggtcgttggcattgccctaggactcttcttcgactatttaccccaccgccccttccaagagcgcaaccgctggcgcaatgcacgggtctatcccggtcaggtgatgaacatcctgatcatgggtcagaactatcacctgatccatcacctctggccatcgatcccctggtatctctaccgaccggcctaccacgctaccaagccgttgttggacctacgccagtcgccgcaaacgctcgggattctctccagcaaaaaagatttctggaactttatctacgacgttttcatcggcatccgcattcaccaatcgcacgaggctgagccgcagagctccgtcgttcctgaaacgaagtcgagtgaatcagccgttctcgcaaaagctccgatgtctgccacagaagactctcgtgagccagccttgacgaagtag

SEQ ID NO: 22 - DNA, 374 bp, Pantoea ananatis
cggcgcatagaggaagccaaaagaaacacaaccttctttgcccctgacggcgtgatgcatacggtgcgccatatacaaccgtttgaggtagcccttgcgtggaatatagcggaatggccaacgttgatgcaccagcccgtcgtgcaccataaaatagagtaatccatacgccgtcatacctgcgccaatccactggagcggccacattcctgtactgcccagataaatcagcaggatcgataatgcagcaaaaaccacggcataaagatcgttaacttcaaacgcacctttacgcggttcatgatgtgaaagatgccatccccaaccccagccgtgcatgatgtatttgtgtgccagtgcagcaatcacttcca

SEQ ID NO: 23 - DNA, 10566 bp, Artificial Sequence
  pTJ001/pMX570 plasmid
agcttgtcatctgccggatgaggcaaaaccctgcctacggcgcgattacatcgtcccagcgcgatcgctcttactgttgatggctcgtgcttaaaaacaatgcaaacttcaccgtttcagctggtgattttcgactgtgatggtgtgcttgttgatagcggaacgcatcactaatcgcgtctttgcagacatgctcaatgaactgggtctgttggtgactttggatgacatgtttgagcagtttgtgggtcattccatggctgactgtctcaaactaattgagcgacggttaggcaatcctccaccccctgactttgttcagcactatcaacgccgtacccgtatcgcgttagaaacgcatctacaagccgttcctggggttgaagaggctttggatgctcttgaattgccctactgtgttgcgtccagtggtgatcatcaaaagatgcgaaccacactgagcctgacgaagctctggccacgatttgagggacgaatcttcagcgtgactgaagtacctcgcggcaagccatttcccgatgtctttttgttggccgccgatcgcttcggggttaatcctacggcctgcgctgtgatcgaagacacccccttgggagtagcggcaggcgtggcggcaggaatgcaagtgtttggctacgcgggttccatgcccgcttggcgtctgcaagaagccggtgcccatctcatttttgacgatatgcgactgctgcccagtctgctccaatcgtcgccaaaagataactccacagcattgcccaatccctaacccctgctcgcgccgcaactacacactaaaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacgggtaaccgatatcgcttgcaattcgcgctaacttacattaattgcgttgcgctcattgaccactctccaaacggctcacttgccgtgccagctgcatgagactatcggccagagcacgcggggaggccgtttgcgtgtttggcgccaaggtggttttgcgtttcaccagcgacacgggcagcagctgatttcctttaactgcctgcccttggctcagttgcaacagtcgatccacggacgtctgacccaacaagcggaaatcttgcttgatcgtggtcagaggcgggatataacaactcgaatcttcagtatcatcatagcccacgacactaatatctgcgccgacgcggaggccgctctccgtgatcgcacgcatcgcgcccaaagccatctggtcattcgcgaccagcatggccgtaggcacgatgccttcattcagcatttgcattgtttgctggaaacccgacatagcgctccaatcgccctcgcgctcggcgattggttggatctgattgcgggtgaggtatttatgccagcccgcgagtcgcaggcgagcactcacgctggagagcgggcctgccaagagagcaatctgctgatggcccagcgcgaccagatgctccacacccaagcgtgtaccgtcctcgtgcgagaagatgatgctattaatgggggtttgatcggagacgtccaggaacaacgcgggaacgttcgtgcaggccgcttcaacagcgatagcatcttggtcatccagcgggtagttgataatcaggcccgacacacgctgagccaggaggttgtggaccgcagctttgcaagcttccacgccactccgttcaaccatggagacgaccacgcttgcccccagttgatccgcacgcgatttaatcgcggcaacaatttgactcggggcgtgcagcgcgagagagctcgtggcaaccccgatcaacaagctctgttttccggccagctgctgcgcgacgcggttggggatataattcagctcggccatcgcagcctcgactttttcacgcgtctttgccgacacatgggaggcttgattaaccacgcgactgacagtttggtagctcacacctgcgtattctgcaacatcatacagcgtgactggcttcacattgaccatcctgaattgactctcttccgggcgctatcatgccataccgcgaaaggttttgcaccattcgatggtgtcaacgtacgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacaggaattacatatgtcagaggctcaaacgcccctgacagtaccgaagaagtttcttggtgctccaggaggcttcaaccccaccgtcgcactcttcttggcaggttatacctgcgcggcgctctcagttttggggtactggtgctggagttggccccactggctatctttccttctgagtgtcacagccttacatttggtaggcaccgtcattcacgatgcctctcataatgtggctcacgccagtcgcattctgaatgcgattttgggacatggcagtgcactattgctgggctttacttttccggtgtttacgcgggttcacctgcaacatcacgcccacgtcaacgatcccaagaacgatcccgaccacatcgtttccacctttgggccgctgtggttgatcgcaccgcgcttcttctatcacgagatctatttcttccagcgccgcctttggaagaaatttgaattactcgaatggttcctcagtcgcgctgtggtcatcggcatctttgcctgcggcgtcaagtttggcttcctgggcttcctgatgaactactggctggctccagccttggtcgttggcattgccctaggactcttcttcgactatttaccccaccgccccttccaagagcgcaaccgctggcgcaatgcacgggtctatcccggtcaggtgatgaacatcctgatcatgggtcagaactatcacctgatccatcacctctggccatcgatcccctggtatctctaccgaccggcctaccacgctaccaagccgttgttggacctacgccagtcgccgcaaacgctcgggattctctccagcaaaaaagatttctggaactttatctacgacgttttcatcggcatccgcattcaccaatcgcacgaggctgagccgcagagctccgtcgttcctgaaacgaagtcgagtgaatcagccgttctcgcaaaagctccgatgtctgccacagaagactctcgtgagccagccttgacgaagtagggatcttacccgtatgatgtccctgattatgcatagtctagacccgggctcgagctagcaagcttggccggatccggccggatccgggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttattgagccgttaccccacctactagctaatcccatctgggcacatccgatggcaagaggcccgaaggtccccctctttggtcttgcgacgttatgcggtattagctaccgtttccagtagttatccccctccatcaggcagtttcccagacattactcacccgtccgccactcgtcagcaaagaagcaagcttagatcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgatctggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgcttacgagctcgtttatcgaccacgatcgcatccaaatccgcgatcgcttcccagtccggcaattcagtctggggcgtccgtttcattaatcctgatcaggcacgaaattgctgtgcgtagtatcgcgcatagcggccagcctctgccaacagcgcatcgtgattgcctgcctcaacaatctggccgcgctccatcaccaagatgcggctggcattacgaaccgtagccagacggtgagcaatgataaagaccgtccgtccctgcatcacccgttctagggcctcttgcaccaaggtttcggactcggaatcaagcgccgaagtcgcctcatccagaattaaaatgcgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 24 - DNA, 9345 bp, Artificial Sequence
  pAM1579Fara3 plasmid
agcttgtcatctgccggatgaggcaaaaccctgcctacggcgcgattacatcgtcccagcgcgatcgctcttactgttgatggctcgtgcttaaaaacaatgcaaacttcaccgtttcagctggtgattttcgactgtgatggtgtgcttgttgatagcggaacgcatcactaatcgcgtctttgcagacatgctcaatgaactgggtctgttggtgactttggatgacatgtttgagcagtttgtgggtcattccatggctgactgtctcaaactaattgagcgacggttaggcaatcctccaccccctgactttgttcagcactatcaacgccgtacccgtatcgcgttagaaacgcatctacaagccgttcctggggttgaagaggctttggatgctcttgaattgccctactgtgttgcgtccagtggtgatcatcaaaagatgcgaaccacactgagcctgacgaagctctggccacgatttgagggacgaatcttcagcgtgactgaagtacctcgcggcaagccatttcccgatgtctttttgttggccgccgatcgcttcggggttaatcctacggcctgcgctgtgatcgaagacacccccttgggagtagcggcaggcgtggcggcaggaatgcaagtgtttggctacgcgggttccatgcccgcttggcgtctgcaagaagccggtgcccatctcatttttgacgatatgcgactgctgcccagtctgctccaatcgtcgccaaaagataactccacagcattgcccaatccctaacccctgctcgcgccgcaactacacactaaaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacgggtaaccgatatcgctaggacagcttaaccgccacgtcattgactttttcttcacaacctgcgcgaaattcagagggcgatgcaccagtgcacttcttgaaaactcgcgagaaatacaactggtcatcaaacccgacattgcggccaacagtagcgatgggcatccgcgtggtggacaagagcagcttggcctgcgaaatccgttgatcttcgcgccaagacaggaccgagataccgagctgttggcgaaacaggtggctcaagcgcgacgggctgagacaaacatgttgcgcgacggacgcgatgtcgaaattgctgtcagccagatgatcgctgatgtactggcatgcttcgcggactcggttatccatcggcgggtggagcgactcgttgatggcttccatccgccgcaacaggagttgttccaacagattgatagcgaggagttcagagtaacggccctcgccttggccggcgttaataatctggccaaacagatcactaaagtgtggttgatgggcttcatcggggcgaaaaaaacccgtattggcaaaaatagaaggccagttcaaccattcgtgccagtaagcccgtggccgaaagtaaacccactggtgataccattcgcgggcctcgggatggcgaccgtagtgatgaatttcaccaggtgggaacagcaaaatgtcaccggggcgacaaacaaactcccgcccttggtttttgacaacgccctgaccacgaatcgtcaagttcaaaatatagcccttcatacccaaaggtcgatcgatgaagaaatccaaatacccattggcttcgataggtgtcaggccggccacgagatgggcattaaaactataacccggcaacaaaggatcattctgagcttcggccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggcgaattccaggaggaattacatatgaggcctagatcttacccgtatgatgtccctgattatgcatagtctagacccgggctcgagctagcaagcttggccggatccggccggatccgggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttattgagccgttaccccacctactagctaatcccatctgggcacatccgatggcaagaggcccgaaggtccccctctttggtcttgcgacgttatgcggtattagctaccgtttccagtagttatccccctccatcaggcagtttcccagacattactcacccgtccgccactcgtcagcaaagaagcaagcttagatcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgatctggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgcttacgagctcgtttatcgaccacgatcgcatccaaatccgcgatcgcttcccagtccggcaattcagtctggggcgtccgtttcattaatcctgatcaggcacgaaattgctgtgcgtagtatcgcgcatagcggccagcctctgccaacagcgcatcgtgattgcctgcctcaacaatctggccgcgctccatcaccaagatgcggctggcattacgaaccgtagccagacggtgagcaatgataaagaccgtccgtccctgcatcacccgttctagggcctcttgcaccaaggtttcggactcggaatcaagcgccgaagtcgcctcatccagaattaaaatgcgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 25 - DNA, 9579 bp, Artificial Sequence
  pAM1579Ftrc3 plasmid with pTrc promoter
agcttgtcatctgccggatgaggcaaaaccctgcctacggcgcgattacatcgtcccagcgcgatcgctcttactgttgatggctcgtgcttaaaaacaatgcaaacttcaccgtttcagctggtgattttcgactgtgatggtgtgcttgttgatagcggaacgcatcactaatcgcgtctttgcagacatgctcaatgaactgggtctgttggtgactttggatgacatgtttgagcagtttgtgggtcattccatggctgactgtctcaaactaattgagcgacggttaggcaatcctccaccccctgactttgttcagcactatcaacgccgtacccgtatcgcgttagaaacgcatctacaagccgttcctggggttgaagaggctttggatgctcttgaattgccctactgtgttgcgtccagtggtgatcatcaaaagatgcgaaccacactgagcctgacgaagctctggccacgatttgagggacgaatcttcagcgtgactgaagtacctcgcggcaagccatttcccgatgtctttttgttggccgccgatcgcttcggggttaatcctacggcctgcgctgtgatcgaagacacccccttgggagtagcggcaggcgtggcggcaggaatgcaagtgtttggctacgcgggttccatgcccgcttggcgtctgcaagaagccggtgcccatctcatttttgacgatatgcgactgctgcccagtctgctccaatcgtcgccaaaagataactccacagcattgcccaatccctaacccctgctcgcgccgcaactacacactaaaccgttcctgcgcgatcgctcttactgttgatggctcgtgcttaaaaacaatgcaaccctaaccgtttcagctggtgattttcggacgatttggcttacagggataactgagagtcaacagcctctgtccgtcattgcacacccatccatgcactggggacttgactcatgctgaatcacatttcccttgtccattgggcgagaggggaggggaatcttctggactcttcactaagcggcgatcgcaggttcttctacccaagcagtggcgatcgcttgattgcagtcttcaatgctggcctctgcagccatcgccgccaccaaagcatcgtaggcgggacgttgttgctccagtaaagtcttcgcccgtaacaatccccagcgactgcgtaaatccgcttcggcaggattgcgatcgagttgccgccacagttgtttccactgggcgcgatcgtcagctcccccttccacgttgccgtagaccagttgctctgccgctgcaccggccatcaacacctgacaccactgttccagcgatcgctgactgagttgcccctgtgcggcttcggcttctagcgcagctgcttggaactgcacacccccgcgaccaggttgtccttggcgcagcgcttcccacgctgagagggtgtagcccgtcacgggtaaccgatatcgcttgcaattcgcgctaacttacattaattgcgttgcgctcattgaccactctccaaacggctcacttgccgtgccagctgcatgagactatcggccagagcacgcggggaggccgtttgcgtgtttggcgccaaggtggttttgcgtttcaccagcgacacgggcagcagctgatttcctttaactgcctgcccttggctcagttgcaacagtcgatccacggacgtctgacccaacaagcggaaatcttgcttgatcgtggtcagaggcgggatataacaactcgaatcttcagtatcatcatagcccacgacactaatatctgcgccgacgcggaggccgctctccgtgatcgcacgcatcgcgcccaaagccatctggtcattcgcgaccagcatggccgtaggcacgatgccttcattcagcatttgcattgtttgctggaaacccgacatagcgctccaatcgccctcgcgctcggcgattggttggatctgattgcgggtgaggtatttatgccagcccgcgagtcgcaggcgagcactcacgctggagagcgggcctgccaagagagcaatctgctgatggcccagcgcgaccagatgctccacacccaagcgtgtaccgtcctcgtgcgagaagatgatgctattaatgggggtttgatcggagacgtccaggaacaacgcgggaacgttcgtgcaggccgcttcaacagcgatagcatcttggtcatccagcgggtagttgataatcaggcccgacacacgctgagccaggaggttgtggaccgcagctttgcaagcttccacgccactccgttcaaccatggagacgaccacgcttgcccccagttgatccgcacgcgatttaatcgcggcaacaatttgactcggggcgtgcagcgcgagagagctcgtggcaaccccgatcaacaagctctgttttccggccagctgctgcgcgacgcggttggggatataattcagctcggccatcgcagcctcgactttttcacgcgtctttgccgacacatgggaggcttgattaaccacgcgactgacagtttggtagctcacacctgcgtattctgcaacatcatacagcgtgactggcttcacattgaccatcctgaattgactctcttccgggcgctatcatgccataccgcgaaaggttttgcaccattcgatggtgtcaacgtacgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacaggaattacatatgaggcctagatcttacccgtatgatgtccctgattatgcatagtctagacccgggctcgagctagcaagcttggccggatccggccggatccgggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttattgagccgttaccccacctactagctaatcccatctgggcacatccgatggcaagaggcccgaaggtccccctctttggtcttgcgacgttatgcggtattagctaccgtttccagtagttatccccctccatcaggcagtttcccagacattactcacccgtccgccactcgtcagcaaagaagcaagcttagatcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgatctggtaaccccagcgcggttgctaccaagtagtgacccgcttcgtgatgcaaaatccgctgacgatattcgggcgatcgctgctgaatgccatcgagcagtaacgtggcaccccgcccctgccaagtcaccgcatccagactgaacagcaccaagaggctaaaacccaatcccgccggtagcagcggagaactacccagcattggtcccaccaaagctaatgccgtcgtggtaaaaatcgcgatcgccgtcagactcaagcccagttcgctcatgcttcctcatctaggtcacagtcttcggcgatcgcatcgatctgatgctgcagcaagcgttttccataccggcgatcgcgccgtcgccctttcgctgccgtggcccgcttacgagctcgtttatcgaccacgatcgcatccaaatccgcgatcgcttcccagtccggcaattcagtctggggcgtccgtttcattaatcctgatcaggcacgaaattgctgtgcgtagtatcgcgcatagcggccagcctctgccaacagcgcatcgtgattgcctgcctcaacaatctggccgcgctccatcaccaagatgcggctggcattacgaaccgtagccagacggtgagcaatgataaagaccgtccgtccctgcatcacccgttctagggcctcttgcaccaaggtttcggactcggaatcaagcgccgaagtcgcctcatccagaattaaaatgcgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 26 - DNA, 816 bp, Synechococcus elongatus PCC 7942
gtgaacctgcttgatcgctctttgagtcctgatagcttgatcttcttcggcctgatcttaggacgttatctactgattgctggaggactctactggctcttttatggctttttggcagagcgatctactcgcaggagagtgaagcattggccgacttggaagaagtcaattcgctcagatgttgggctttcgatagcctcggctttagtctttgccctattagcttcttggttgacttctactccagtgctgatccatacgcggctctacacagatgttagtcaatatggatttttttatttaatctttagcttctttctggttctgattctgcaagatactagtttttatttctgtcatcgactctttcatcagccttggttatttcgctgggtacatcggggtcatcatcgttcgcaatgcccaacaccctggacctcctttgcctttgatctacccgaagcagtgatgcaggcactgctgattattggcattgtttttgttttcccagttcacgttggcattttgctggctgccttggtgactatgaccctttggtctgttttcaaccatttgggcttctcactgttccccgattcaggggtttctcagtggctggctcagtggttgatcggaccccagcatcatctgcttcaccatcaacgctacagctttcactacggtctgtacttcacgttttgggatcgactcctaggcactcaaattccagcctcccagccgcagactcaacgctcggttcgtctggctttactcactgctgcaaaacccttggcagtccgaggtaaagaacgctaa

SEQ ID NO: 27 - DNA, 1221 bp, Artificial Sequence
  Kanamycin resistance gene
ctgtctcttatacacatctcaaccatcatcgatgaattgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcctctagagtcgacctgcaggcatgcaagcttcagggttgagatgtgtataagagacag

SEQ ID NO: 28 - DNA, 534 bp, Sphingomonas sp. LH128
atgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaa

SEQ ID NO: 29 - DNA, 735 bp, Brevundimonas sp. SD212
atgaccgccgccgtcgccgagccccgcatcgtcccgcgccagacctggatcggtctgaccctggcgggaatgatcgtggcgggatgggggagcctgcacgtctacggcgtctattttcaccgctggggcacctccagtctggtgatcgtcccggcgatcgtagcggtccagacctggttgtcggtcggccttttcatcgtcgcccatgacgccatgcacggctccctggcgccgggacggccgcggctgaacgccgcagtcggccggctgaccctggggctctatgcgggcttccgcttcgatcggctgaagacggcgcaccacgcccaccacgccgcgcccggcacggccgacgacccggacttttacgccccggcgccccgcgccttccttccctggttcctgaacttctttcgcacctatttcggctggcgcgagatggcggtcctgaccgccctggtcctgatcgccctcttcggcctgggggcgcggccggccaatctcctgaccttctgggccgcgccggccctgctttcagcgcttcagctcttcaccttcggcacctggctgccgcaccgccacaccgaccagccgttcgccgacgcccaccacgcccgcagcagcggctacggccccgttctttccctgctcacctgcttccacttcggccgccaccacgaacaccacctcaccccctggcggccctggtggcgtttgtggcgcggcgagtcttga

SEQ ID NO: 30 - DNA, 7238 bp, Artificial Sequence
  pS1s-Ptrc-crtW plasmid
cgcgccatcgcttgcaattcgcgctaacttacattaattgcgttgcgctcattgaccactctccaaacggctcacttgccgtgccagctgcatgagactatcggccagagcacgcggggaggccgtttgcgtgtttggcgccaaggtggttttgcgtttcaccagcgacacgggcagcagctgatttcctttaactgcctgcccttggctcagttgcaacagtcgatccacggacgtctgacccaacaagcggaaatcttgcttgatcgtggtcagaggcgggatataacaactcgaatcttcagtatcatcatagcccacgacactaatatctgcgccgacgcggaggccgctctccgtgatcgcacgcatcgcgcccaaagccatctggtcattcgcgaccagcatggccgtaggcacgatgccttcattcagcatttgcattgtttgctggaaacccgacatagcgctccaatcgccctcgcgctcggcgattggttggatctgattgcgggtgaggtatttatgccagcccgcgagtcgcaggcgagcactcacgctggagagcgggcctgccaagagagcaatctgctgatggcccagcgcgaccagatgctccacacccaagcgtgtaccgtcctcgtgcgagaagatgatgctattaatgggggtttgatcggagacgtccaggaacaacgcgggaacgttcgtgcaggccgcttcaacagcgatagcatcttggtcatccagcgggtagttgataatcaggcccgacacacgctgagccaggaggttgtggaccgcagctttgcaagcttccacgccactccgttcaaccatggagacgaccacgcttgcccccagttgatccgcacgcgatttaatcgcggcaacaatttgactcggggcgtgcagcgcgagagagctcgtggcaaccccgatcaacaagctctgttttccggccagctgctgcgcgacgcggttggggatataattcagctcggccatcgcagcctcgactttttcacgcgtctttgccgacacatgggaggcttgattaaccacgcgactgacagtttggtagctcacacctgcgtattctgcaacatcatacagcgtgactggcttcacattgaccatcctgaattgactctcttccgggcgctatcatgccataccgcgaaaggttttgcaccattcgatggtgtcaacgtacgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaattgtcgacaggaaacaggaattacatatgacggctgcggtcgctgagccgcgaatcgtgcctcgtcaaacctggatcgggctcacacttgcagggatgattgtggcaggctgggggagtcttcacgtctatggagtctactttcatcgctgggggacaagtagcctcgttattgtgccagcaatcgtggctgtccagacctggctcagtgtgggcctattcattgtcgcacacgacgctatgcacgggtcgctggcgccgggtcggcctcgcctaaacgcagccgtaggcagactcaccttagggctctatgccggtttccgatttgatcgccttaaaacggcccaccacgcacatcacgccgcccccggtactgccgatgaccctgatttctacgccccagcaccgcgagcgtttttgccttggttcctcaacttctttcgcacttactttggctggcgtgagatggctgttctgacagctttagtcctaatagccttgtttggtttgggtgcccggccggcaaatcttctcacgttctgggcagctccggcgctgttaagtgctctacagctattcacttttggcacgtggctgccgcaccgccacactgatcagccttttgcggacgcccaccatgctcggtcttccggttacggcccggtgctcagcctgcttacctgttttcactttggtcggcaccacgaacaccatctgacaccgtggcgcccctggtggcggctatggcgaggtgagagcagatctagtctcgagcaaaagctaattagcgaggaagacctagaacaaaaactgatctctgaagaggatctggagcagaagttgatttcggaggaagacttgtagactagtcatcgagctagcaagcttggccggatccggccggatccggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcggtacccgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcgaattcacatacgcggccgcctgggccttgagctcgaatttcgagcttctggagcaggaagatgtcgcgggcattagcaccagcggtctgccaagcctccgccagccgttgggtcccttccgcttgagcttttccatcttcgacgatacgggcggcggccccccgcgcttccgcgatcgcccgtttacaagctgcctcagctggggcgatcacatcggcttgaagttgctgctgcacctgtttgatccgctcctgctgcacagggagttctgcttggctacgagcgacttcggtagcaatgtccgcttcagcttcggccaccaccgcttcgcgccgcgtcaacgcatcctgaatccggcgctcggcctcggcttgggcgatcgctacatcgcgatcgatccgacgcagggccgtgatcttgtcattttcggccgtttggatcgcagaggcagcctgggcatcggcttcagcaattcgggcatctcgctgcagatcagcccgctgcttgcgtccactagccgagagataaccgacctcatcgatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatggacatattgtcgttagaacgcggctacaattaatacataaccttatgtatcatacacatacgatttaggtgacactatagaagcccagaggtgatgaactcgacgaggccattgaagaaggggcgctcgcggtggacagcatagtgctgttcggccagctcgcgactgggcttcagctgctttaggcccaccagtttgaagcctttttgctcaaagcggccgatgatcgtaccgaccaaaccccgctgaacgccatcgggcttgatggcaataaatgtgcgttccacagacatctagatagtcctcaagacgaggcaagcattgagcttgccttcctatggttcgggatcactgggattcttgacaagcgatcgcggtcacatcgctatctcttaggacttcgcagcgggcgagtcggattgacccggtagggatttcgccagatcaatgcccgtggtttgtttcagcttctccagcaagctagcgatttgggtagcgctgccttccccttcgccaatcacagtgatcgactccacgtcgatatctggcacggtgcctgaaagcgtgactagcagggactcgaagcttgcatgcctgcaggtgg

(19) United States
(12) Patent Application Publication
(10) Pub. No.: US 2017/0240944 A1
(43) Pub. Date: Aug. 24, 2017

(54) Modified Cyanobacteria for Producing Carotenoids

(71) Applicant: MATRIX GENETICS, LLC, Seattle, WA (US)

(72) Inventors: Mark Heinnickel, Seattle, WA (US); James Roberts, Seattle, WA (US)

(21) Appl. No.: 15/519,499

(22) PCT Filed: Oct. 14, 2015
(86) PCT No.: PCT/US15/55601

§ 371 Date: Apr. 14, 2017

Related U.S. Application Data
(60) Provisional application No. 62/063,865, filed on Oct. 14, 2014.

(51) Int. Cl. C12P 23/00; C12N 15/82; C12N 1/12

(52) U.S. Cl. CPC C12P 23/00; C12Y 117/05; C12N 1/12; C12Y 114/13129; C12N 15/8222

(57) ABSTRACT

This disclosure describes modified photosynthetic microorganisms, including Cyanobacteria that produce carotenoids, including zeaxanthin, astaxanthin, and/or canthaxanthin. The modifications include one or more genetic modifications such as gene deletion, up regulation of an endogenous gene, and/or addition of an exogenous gene. In some embodiments the modified photosynthetic microorganisms may be subjected to stress conditions.

### PRIORITY CLAIM

This application is a national stage application of international patent application No. PCT/US15/55601, entitled “Modified Cyanobacteria For Producing Carotenoids,” and filed Oct. 14, 2015, which claims priority to U.S. Provisional Patent Application No. 62/063,865, entitled “Modified Cyanobacteria for Producing Carotenoids,” and filed Oct. 14, 2014. PCT International Application No. PCT/US15/55601 and U.S. Provisional Application Ser. No. 62/063,865 are fully incorporated herein by reference in their entirety.

### SEQUENCE LISTING

The Sequence Listing associated with this application is provided in text format in lieu of a paper copy, and is hereby incorporated by reference into the specification. The name of the text file containing the Sequence Listing is M077-0015PCT Sequence Listing.txt. The text file is about 112 KB, was created on Oct. 9, 2015, and is being submitted electronically via EFS-Web.

### BACKGROUND

Certain cyanobacteria can be utilized as a source of carotenoids. Carotenoids can be produced from fats and other basic organic metabolic building blocks by these photosynthetic organisms. Carotenoids function in photosynthesis to protect components of the photosynthetic apparatus from oxidative stress. Carotenoids may also provide various yellow to red shades of pigmentation.

Cyanobacteria, also known as blue-green algae, blue-green bacteria, or Cyanophyta, are a phylum of bacteria that obtain energy from photosynthesis, utilizing chlorophyll a and water to reduce CO₂. Certain Cyanobacteria can produce metabolites, such as carotenoids, from just sunlight, CO₂, water, and inorganic salts. Unlike many other photosynthetic organisms, Cyanobacteria can be genetically manipulated. For example, Synechococcus elongatus PCC 7942 (hereafter referred to as “S. elongatus PCC 7942”) is a genetically manipulable, unicellular, Cyanobacteria that is very widespread in the marine environment. In the wild S. elongatus PCC 7942 accumulates carotenoids to about 0.47% by dry weight. Cyanobacteria naturally synthesize carotenoids. However, the types of carotenoids synthesized may not be the most commercially valuable and the amount of carotenoids synthesized may be low.

Clearly, therefore, there is a need for modified Cyanobacteria capable of producing carotenoids to be used for human and animal consumption and/or in the synthesis of various specialty chemicals.

### BRIEF SUMMARY

This disclosure describes modified Cyanobacteria that produce carotenoids, including, but not limited to, zeaxanthin, astaxanthin, and/or canthaxanthin. The modifications include one or more genetic modifications such as gene deletion, up regulation of endogenous genes, and/or addition of exogenous genes. In some embodiments the modified photosynthetic microorganisms may be subjected to stress conditions. In some embodiments the carotenoids may be excreted by the modified Cyanobacteria into the media. Embodiments of the present invention relate to the demonstration that Cyanobacteria can be modified to increase synthesis of carotenoids and to drive synthesis towards production of specific carotenoids including carotenoids not naturally found in a corresponding wild-type organism.

In some embodiments, the present disclosure includes a method for producing a carotenoid by culturing modified cyanobacteria having deletion of a glycogen pathway gene or addition of a regulatable, exogenous promoter that controls transcription of a glycogen pathway gene and subjecting the modified cyanobacteria to a stress condition during culturing. When subjected to the stress condition the modified cyanobacteria produce the carotenoid at a level greater than the corresponding wild-type photosynthetic microorganisms. Furthermore, the Cyanobacteria modified with the deletion of a glycogen pathway gene produce the carotenoid at a greater rate than a Cyanobacteria (either WT or with other genetic modifications) without deletion of the glycogen pathway gene. In an embodiment, the carotenoid is produced by generating modified Cyanobacteria by addition of a polynucleotide encoding a carotenoid hydroxylase and a promoter or by a modification that increases synthesis of a carotenoid hydroxylase from one or more endogenous genes. Increase in levels of carotenoid hydroxylase may drive synthesis of zeaxanthin. In an embodiment, the present disclosure includes modification of cyanobacteria by addition of an exogenous polynucleotide encoding a carotenoid ketolase. In embodiments in which a gene encoding the carotenoid ketolase is added to cyanobacteria that do not naturally synthesize canthaxanthin and astaxanthin, the cyanobacteria may synthesize canthaxanthin and astaxanthin as a result of the modification.

In some embodiments, the present disclosure includes modified cyanobacteria that comprise an exogenous polynucleotide encoding a carotenoid ketolase and a deleted glycogen pathway gene. When subjected to a stress condition, the modified cyanobacteria produce one or more carotenoids at levels greater than the corresponding wild-type cyanobacteria.

In some embodiments, the present disclosure includes methods for generating modified cyanobacteria by deleting a glycogen pathway gene or by adding a regulatable, exogenous promoter that controls transcription of the glycogen pathway gene and by adding an exogenous gene involved in the carotenoid biosynthesis pathway. The modified cyanobacteria produce a carotenoid at a level greater than the corresponding wild-type photosynthetic microorganisms. In embodiments, the gene involved in the carotenoid biosynthesis pathway may be a carotenoid ketolase gene or a carotenoid hydroxylase gene. In an embodiment, the regulatable, exogenous promoter controls transcription of the one of more glycogen pathway genes and the promoter is activated by a nutrient the absence of which creates a stress condition for the modified cyanobacteria.

In embodiments of the invention, the Cyanobacteria may be Synechococcus, Synechocystis, or Spirulina. In an embodiment the Synechococcus may be S. elongatus PCC 7942.

In embodiments of the invention, the glycogen pathway gene may be glgC that encodes glucose-1-phosphate adenylyltransferase.

In embodiments of the invention, the carotenoid ketolase gene may be crtW which encodes a β-carotene oxygenase such as Brevundimonas sp. SD212 β-carotene oxygenase.

In embodiments of the invention, the carotenoid hydroxylase gene may be crtR which encodes β-carotene hydroxylase such as Synechococcus β-carotene hydroxylase, crtG which encodes 2, 2′β-hydroxylase, or crtZ which encodes a 3,3′-hydroxylase that hydroxylates canthaxanthin.

In embodiments of the invention, the carotenoid may be a xanthophyll such as zeaxanthin, astazanthin, or canthaxanthin.

In embodiments of the invention, the stress condition may be high light, high salt, nitrogen deprivation, sulfur deprivation, phosphorous deprivation, or iron deprivation.

In an embodiment of the invention, the level of carotenoid production may be at least about five times greater than the carotenoid production of the corresponding wild-type cyanobacteria. In an embodiment of the invention, the higher carotenoid level as compared to the corresponding wild-type photosynthetic microorganisms may be reached by about 24 48, 72, 96, 144, or 168 hours after initiation of the stress condition.

### BRIEF DESCRIPTION OF THE FIGURES

FIG. 1 shows a carotenoid biosynthesis pathway in S. elongatus sp. that includes exogenous enzymes and modified endogenous enzymes.

FIG. 2 shows thin-layer chromatograph (TLC) separation of carotenoids comparing a wild-type Synechococcus to genetically modified Synechococcus that has a crtG deletion. Removal of crtG leads to increased synthesis of zeaxanthin and a complete removal of both caloxanthin and nostoxanthin without harming exponential growth of the Synechococcus cells.

FIGS. 3A and 3B are charts showing changes in the dry weight of five carotenoids following deletion of crtG in Synechococcus. Deletion of crtG leads to increased accumulation of zeaxanthin and decreased or unchanged levels of the other four carotenoids.

FIG. 4 is a chart showing levels of absorbance of light between 350 nm and 800 nm wavelengths. Higher absorption indicates higher levels of carotenoids. Whole cells were examined by spectrophotometry and absorbance as a function of wavelength was determined. The three major peaks represent absorption by chlorophyll A (at approximately 450 and 680 nm) and by phycobiliprotein (at approximately 620 nm). A strain of Synechococcus without modification of the glgC gene (wild-type) and a strain of Synechococcus with deletion of the glgC gene (ΔglgC) are both measured under replete conditions and under nitrogen starvation. The combination of glgC deletion and nitrogen starvation results in a large increase in carotenoid levels.

FIG. 5 is a chart showing increase in carotenoid content of wild-type Synechococcus, Synechococcus with a deletion of the glgC gene, and Synechococcus with deletion of both the glgC and crtG genes under nitrogen stress as compared to wild-type Synechococcus with sufficient nitrogen.

FIGS. 6A and 6B are charts showing changes in the carotenoid content of five carotenoids when a strain of Synechococcus with a glgC deletion is subjected to nitrogen starvation. With this deletion to the glycogen synthesis pathway, the carotenoid content overall increases by about five times under nitrogen starvation as compared to the same mutant in replete conditions. Carotenoid content is measured in mg/OD-mL by optical density readings of liquid culture in 1 cm×1 cm cuvettes at 750 nm. Blank measurements are performed on sterile media. Weigh in mg is derived from the measured optical density.

FIG. 7 is a chart showing estimated dry weight of carotenoids in wild-type and glgC deleted Synechococcus under replete, nitrogen starvation, sulfur starvation, and phosphorous starvation conditions. The dry weight of carotenoids increases to about 1.2% of total dry weight for a strain of Synechococcus with glgC deleted and placed under nitrogen starvation.

FIG. 8 shows the pTJ001 plasmid with crtR regulated by the pTrc promoter and genes for spectinomycin and streptomycin resistance.

FIG. 9 shows TLC separation of carotenoids obtained from Synechococcus with glgC deleted and addition of exogenous crtW in comparison with a canthaxanthin standard. The samples from the modified Synechococcus include bands that correspond to canthaxanthin.

FIG. 10 shows liquid chromatography-mass spectrometry (LC-MS) chromatograms of a canthaxanthin standard (top) and Synechococcus with glgC deleted and addition of exogenous crtW (bottom) in the presence of 1 mM isopropyl β-D-1-thiogalactopyranoside (IPTG). The retention time of the standard canthaxanthin is the same as the major peak in the modified Synechococcus sample. Both samples also have the same molecular mass of 565 as shown in the inset charts.

FIG. 11 shows LC-MS chromatograms of Synechococcus with glgC deleted and addition of exogenous crtW (top) without induction of crtW and Synechococcus with glgC deleted and addition of exogenous crtW (bottom) in the presence of 1 mM IPTG. Induction of crtW expression by addition of IPTG (the change from the top chromatogram to the bottom chromatogram) results in the largest peak shifting from 14.62 min (zeaxanthin) to 16.38 min (canthazanthin). The inset at the top shows the absorption spectrum of a zeaxanthin standard (14.62 min) and the bottom inset shows the absorption spectrum of a canthaxanthin standard (16.38 min).

FIG. 12 shows a growth curve of Synechococcus with glgC deleted and addition of exogenous crtW as measured at 750 optical density with crtW induced, upper line, by the presence of IPTG and crtW not induced, lower line. Expression of crtW does not significantly affect growth.

FIG. 13 shows absorbance from 350 nm to 800 nm of the pTrc-crtW/ΔglgC mutant uninduced under standard conditions, induced under low iron conditions, and induced under high iron conditions. Chlorohpyll levels represented by absorbance at 680 nm decreased under low iron conditions, but were similar for both the uninduced/standard conditions and induced/high iron conditions.

FIG. 14 shows TLC separation of carotenoids comparing carotenoids produced by the pTrc-crtW-crtZ/ΔglgC mutant with an induced crtW-crtZ operon to an astaxanthin standard. The modification of Synechococcus combined with the induction of crtW and crtZ leads to production of astaxanthin.

FIG. 15 shows a chromatogram (top) of absorbance at 550 nm of the elution profile of carotenoids from the pTrc-crtW-crtZ/ΔglgC mutant induced with IPTG. This profile includes a large peak with an elution time of 13.46 min. This large peak has the same absorbance spectrum, mass, and retention time as astaxanthin.

FIG. 16 is a chart showing increase in canthaxathin production resulting from inducing crtW in the pTrc-crtW/ΔglgC strain and additional increases in canthaxathin under stress from nitrogen deprivation and high light.

FIG. 17 shows the amount of astaxanthin produced as a result of the induction of crtW-crtZ in the pTrc-crtW-crtZ/ΔglgC strain. Astaxanthin is retained within the cell and excreted into the media.

FIG. 18 shows the pAM1579Fara3 plasmid with the pBAD promoter and kanamycin resistance.

FIG. 19 shows the pAM1579Ftrc3 plasmid with the pTrc promoter and kanamycin resistance.

FIG. 20 shows the pS1s-pTrc-crtW plasmid with crtW under control of the pTrc promoter and resistance to spectinomycin and streptomycin.

FIG. 21 shows the pMX5-SmR-pTrc-crtW-crtZ plasmid with the pTrc promoter resistance to spectinomycin and streptomycin.

FIG. 22 shows a growth curve of six samples of Synechococcus measured at OD 750. The six samples include three strains all with addition of crtW and crtZ genes. Two of the strains additionally have a crtG deletion. Two of the strains additional have a glgC deletion. For each strain a sample was generated with crtW induced by addition of IPTG and without induction of crtW. The combination of addition of crtW (induced), addition of crtZ, and deletion of glgC lead to the highest level of growth.

FIG. 23 is a chart showing astaxanthin levels present in the six samples evaluated in FIG. 22. The combination of addition of crtW (induced), addition of crtZ, and deletion of glgC resulted in the highest level of astaxanthin synthesis.

### DETAILED DESCRIPTION

The present disclosure relates generally to modified Cyanobacteria and methods of generation thereof, which have been modified to produce or store increased levels of carotenoids as compared to wild-type cyanobacteria.

The present disclosure relates, in part, to the discovery that carotenoid biosynthesis in Cyanobacteria can be modified by reducing the expression of certain genes, increasing the expression of certain genes, and/or introducing exogenous genes. For instance, Cyanobacteria which contains deletions of crtG (2,2′β-carotene hydroxylase) leads to increased synthesis and accumulation of zeaxanthin while maintaining typical rates of exponential growth. Additionally, Cyanobacteria which have overexpression of crtR (3,3′β-carotene hydroxylase) or crtZ (cartonenoid-3,3′-hydroxylase that can hydroxylate canthaxanthin) also have increased synthesis and accumulation of zeaxanthin as compared to the wild-type Cyanobacteria. Additionally, introduction of crtW (β-carotene oxygenase) and crtZ (cartonenoid-3,3′-hydroxylase that can hydroxylate canthaxanthin) leads to synthesis and accumulation of astaxanthin and canthaxanthin in strains of Cyanobacteria that do not naturally produce either astaxanthin or canthaxanthin.

The present disclosure relates, in further part, to the discovery that reducing expression of certain genes involved in glycogen synthesis combined with placing Cyanobacteria under a stress condition leads to increased synthesis and accumulation of carotenoids. For instance, Cyanobacteria which contain deletions or down-regulation of glgC (glucose-1-phosphate adenylyltransferase) have increased carotenoid synthesis when subjected to nutrient deprivation such as nitrogen deprivation.

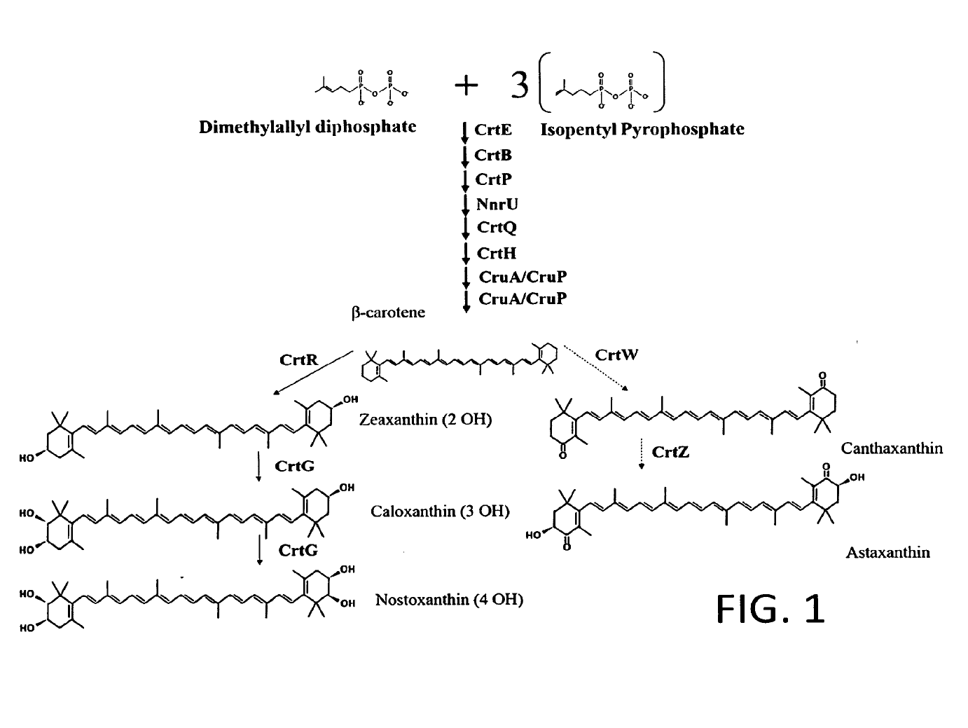

FIG. 1 shows a biochemical pathway in S. elongatus PCC 7942 from carotenoid precursors dimethylallyl diphosphate (DMAPP) and isopentenyl pyrophosphate (IPP) to astaxanthin. Neither astaxanthin nor cantaxanthin are found in any natural cyanobacterial strains. This disclosure describes how the economically valuable carotenoid astaxanthin can be produced in cyanobacteria from β-carotene by the enzymes CrtW and CrtZ, carotenoid ketolase and keto-carotenoid hydroxylase, respectively. In this pathway the CrtW enzyme, an iron dioxygenase, places carbonyl groups on 6 carbon rings of the β-carotene molecule. The resulting ketone species, known as canthaxanthin, can be hydroxylated by the enzyme CrtZ to make astaxanthin. It has been hypothesized that CrtW can ketolate zeaxanthin to astaxanthin directly, but this reaction has been found in vitro to proceed at a much slower rate.

Exogenous proteins are CrtW and CrtZ. Enzymes coded for by the S. elongatus native genes are CrtR and CrtG. The enzymes that are encoded by genes that are native to Brevundimonas sp. Strain SD212 are indicated in red (CrtW and CrtZ). The steps that these exogenous proteins catalyze are indicated by dashed lines (oxygenation of β-carotene and hydroxylation of canthaxanthin).

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, exemplary methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As used herein, the terms “having,” “has,” “contain,” “including,” “includes,” “include,” and “have” have the same open-ended meaning as “comprising,” “comprises,” and “comprise” provided above.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “about” is meant a quantity, level, value, number, frequency, percentage, dimension, size, amount, weight or length that varies by as much as 30, 25, 20, 15, 10, 9, 8, 7, 6, 5, 4, 3, 2, or 1% to a reference quantity, level, value, number, frequency, percentage, dimension, size, amount, weight or length.

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds) and provided literal support for and includes the end points of 10 and 100.

The present description uses specific numerical values to quantify certain parameters relating to the invention, where the specific numerical values are not expressly part of a numerical range. It should be understood that each specific numerical value provided herein is to be construed as providing literal support for a broad, intermediate, and narrow range. The broad range associated with each specific numerical value is the numerical value plus and minus 60 percent of the numerical value, rounded to two significant digits. The intermediate range associated with each specific numerical value is the numerical value plus and minus 30 percent of the numerical value, rounded to two significant digits. The narrow range associated with each specific numerical value is the numerical value plus and minus 15 percent of the numerical value, rounded to two significant digits. These broad, intermediate, and narrow numerical ranges should be applied not only to the specific values, but should also be applied to differences between these specific values.

As used herein “carotenoid” means an organic pigment having the formula C40H56, is formed from eight isoprene units, and includes a series of conjugated double bonds. Carotenoids are found in the chloroplasts and chromoplasts of plants and some other photosynthetic organisms, including some bacteria and some fungi. There are over 600 known carotenoids; they are split into two classes, xanthophylls (which contain oxygen) and carotenes (which are purely hydrocarbons, and contain no oxygen).

As used herein, “secretion” means that a substance is transported out of a cell and is then localized extracellularly, i.e., in the medium outside the cell (extracellular space).

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

As used herein “upregulate” designates a process that occurs within a cell triggered by a signal or chemical (originating internal or external to the cell), which results in increased expression of one or more genes and as a result the protein(s) encoded by those genes as compared to the expression of the cell absent the signal. Signals may include environmental signals (e.g., nutrient deprivation) as well as chemical signals (e.g., presences of lactose).

As used here “downregulate” designates a process resulting in a cell decreasing expression of one or more genes and resulting protein(s) in response to a signal. The decrease is compared to the expression of the cell absent the signal. The decrease may be partial or a complete stop to detectable levels of gene expression and resultant protein(s).

The recitation “polynucleotide” or “nucleic acid” as used herein designates mRNA, RNA, cRNA, rRNA, cDNA, or DNA. The term typically refers to polymeric form of nucleotides of at least 10 bases in length, either ribonucleotides or deoxynucleotides or a modified form of either type of nucleotide. The term includes single and double stranded forms of DNA and RNA.

As used herein, the terms “DNA” and “polynucleotide” and “nucleic acid” include a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

With regard to polynucleotides, the term “exogenous” refers to a polynucleotide sequence that does not naturally occur in a wild-type cell or organism, but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally occurring polynucleotide sequences that may be found in a given wild-type cell or organism. A vector, plasmid, or other man-made construct that includes an endogenous polynucleotide sequence combined with polynucleotide sequences of the unmodified vector etc. is, as a whole, an exogenous polynucleotide and may also be referred to as an exogenous polynucleotide including an endogenous polynucleotide sequence. For example, certain cyanobacterial species do not typically contain a crtW gene, and, therefore, do not comprise an “endogenous” polynucleotide sequence that encodes a β-carotene ketolase polynucleotide. Also, a particular polynucleotide sequence that is isolated from a first organism and transferred to second organism by molecular biological techniques is typically considered an “exogenous” polynucleotide with respect to the second organism.

Polynucleotides may comprise a native sequence (e.g., an endogenous sequence that encodes protein described herein) or may comprise a variant or fragment, or a biological functional equivalent of such a sequence. Polynucleotide variants may contain one or more substitutions, additions, deletions and/or insertions, as further described herein, preferably such that the enzymatic activity of the encoded polypeptide is not substantially diminished relative to the unmodified or reference polypeptide. The effect on the enzymatic activity of the encoded polypeptide may generally be assessed as described herein and known in the art.

As will be understood by those skilled in the art, the polynucleotide sequences of this disclosure can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

Polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

“Homology” refers to the percentage number of amino acids that are identical or constitute conservative substitutions. Homology may be determined using sequence comparison programs such as GAP which is incorporated herein by reference. In this way sequences of a similar or substantially different length to those cited herein could be compared by insertion of gaps into the alignment, such gaps being determined, for example, by the comparison algorithm used by GAP. Genes described herein also include sequences with at least 90% sequence identity; at least 91% sequence identity; at least 92% sequence identity; at least 93% sequence identity, at least 94% sequence identity, at least 95% sequence identity, at least 96% sequence identity, at least 97% sequence identity, at least 98% sequence identity or at least 99% sequence identity. Percentage of sequence identity is determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions. Exemplary nucleotide sequences that encode the proteins and enzymes of the application encompass full-length reference polynucleotides, as well as portions of the full-length or substantially full-length nucleotide sequences of these genes or their transcripts or DNA copies of these transcripts. Portions of a nucleotide sequence may encode polypeptide portions or segments that retain the biological activity of the reference polypeptide.

By “enzyme reactive conditions” it is meant that any necessary conditions are available in an environment (i.e., such factors as temperature, pH, and lack of inhibiting substances) which will permit the enzyme to function. Enzyme reactive conditions can be either in vitro, such as in a test tube, or in vivo, such as within a cell.

As used herein, the terms “function” and “functional” and the like refer to a biological, enzymatic, or therapeutic function.

The term “biologically active fragment,” as applied to fragments of a reference polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence. The term “reference sequence” refers generally to a nucleic acid coding sequence, or amino acid sequence, of any enzyme having, e.g., glucose-1-phosphate adenylyltransferase activity, β-carotene oxygenase activity, carotenoid ketolase activity, and/or β-carotene hydroxylase activity as described herein.

Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600, or more contiguous nucleotides or amino acid residues in length, including all integers in between, which comprise or encode a polypeptide having an enzymatic activity of a reference polynucleotide or polypeptide. Representative biologically active fragments generally participate in an interaction, e.g., an intra-molecular or an inter-molecular interaction. An inter-molecular interaction can be a specific binding interaction or an enzymatic interaction. Examples of enzymatic interactions or activity include glucose-1-phosphate adenylyltransferase activity, β-carotene oxygenase activity, carotenoid ketolase activity, and/or β-carotene hydroxylase activity, as described herein.

The recitation polypeptide “variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues. Biologically active variants of a reference polypeptide will have at least 40%, 50%, 60%, 70%, generally at least 75%, 80%, 85%, usually about 90% to 95% or more, and typically about 97% or 98% or more sequence similarity or sequence identity to the amino acid sequence for a reference protein as determined by sequence alignment programs described elsewhere herein using default parameters. A biologically active variant of a reference polypeptide may differ from that protein generally by as much 200, 100, 50 or 20 amino acid residues or suitably by as few as 1-15 amino acid residues, as few as 1-10, such as 6-10 amino acid residues, including about 20, 19, 18, 17, 16, 15, 14, 13, 12, 11, 10, 9, 8, 7, 6, 5, 4, 3, 2, or even 1 amino acid residues.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “isolated” is meant material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

By “increased” or “increasing” is meant the ability of one or more modified photosynthetic microorganisms, e.g., Cyanobacteria, to produce a greater amount of a given carotenoid as compared to a control Cyanobacteria, such as an unmodified Cyanobacteria or a differently modified Cyanobacteria. Production of carotenoids can be measured according to techniques known in the art, such as measuring carotenoid concentration per cell with liquid chromatography-mass spectrometry (LC-MS) and comparing results to known standards with concentrations determined with published molar extinction coefficients. The dry weight of carotenoids in cyanobacteria cells can be determined by weighing lyophilized cell pellets.

By “obtained from” is meant that a sample such as, for example, a polynucleotide or polypeptide is isolated from, or derived from, a particular source, such as a desired organism or a specific tissue within a desired organism. “Obtained from” can also refer to the situation in which a polynucleotide or polypeptide sequence is isolated from, or derived from, a particular organism or tissue within an organism. For example, a polynucleotide sequence encoding glucose-1-phosphate adenylyltransferase, β-carotene oxygenase, β-carotene ketolase, or β-carotene hydroxylase may be isolated from a variety of prokaryotic or eukaryotic organisms, or from particular tissues or cells within certain eukaryotic organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO₂ concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., nitrogen, lactose, mannitol, an amino acid), light (e.g., wavelength specific), osmotic potential (e.g., salt induced), a heavy metal, or an antibiotic. Numerous standard inducible promoters will be known to one of skill in the art.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

“Transformation” refers to the permanent, heritable alteration in a cell resulting from the uptake and incorporation of foreign DNA into the host-cell genome; also, the transfer of an exogenous gene from one organism into the genome of another organism.

By “vector” is meant a polynucleotide molecule, preferably a DNA molecule derived, for example, from a plasmid, bacteriophage, yeast, or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably one which is operably functional in a bacterial cell, such as a cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The recitations “mutation” or “deletion,” in this context refer generally to those changes or alterations in the genome of an organism that render the product of a gene non-functional or having reduced function. Examples of such changes or alterations include nucleotide substitutions, deletions, or additions/insertions to the coding or regulatory sequences of a targeted gene (e.g., glgA, glgC, crtG), in whole or in part, which disrupt, eliminate, down-regulate, or significantly reduce the expression of the polypeptide encoded by that gene, whether at the level of transcription, translation, post-translational modification, or protein stability. Such alterations can also reduce the enzymatic activity or other functional characteristic of the protein (e.g., localization), with or without reducing expression.

The terms “wild-type” and “naturally occurring” are used interchangeably to refer to an organism, gene, or gene product that has the characteristics of that organism, gene or gene product (e.g., a polypeptide) when isolated from a naturally occurring source. A wild-type organism, gene, or gene product is that which is most frequently observed in a population and is thus arbitrarily designed the “normal” or “wild-type” form.

All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in this application, the definition provided in this application shall control.

### Photosynthetic Microorganisms

According to an embodiment, a photosynthetic microorganism is genetically modified, for instance, relative to the wild-type or most frequently observed photosynthetic microorganism of that same species. Genetic modifications can be man-made and/or naturally-occurring, for instance, by direct molecular biological intervention (e.g., knocking out of genes, knocking in of genes, cloning or insertion of exogenous genetic elements to modulate expression of genes), directed evolution under controlled conditions to enhance natural selection, or identification of spontaneous mutants under natural conditions, including combinations thereof. For instance, Cyanobacteria, such as Synechococcus, which naturally synthesize various carotenoids, may be modified to produce and accumulate significantly higher levels of carotenoids in general, higher levels of a specific carotenoids, or an exogenous carotenoid that is naturally produced only in other species.

Photosynthetic organisms that may be modified according to the present disclosure may be any type of photosynthetic microorganism. These include, but are not limited to photosynthetic bacteria, green algae, and Cyanobacteria. The photosynthetic microorganism can be, for example, a naturally photosynthetic microorganism, such as a Cyanobacterium, or an engineered photosynthetic microorganism, such as an artificially photosynthetic bacterium. Exemplary microorganisms that are either naturally photosynthetic or can be engineered to be photosynthetic include, but are not limited to, bacteria; fungi; archaea; protists; eukaryotes, such as a green algae; and animals such as plankton, planarian, and amoeba. Examples of naturally occurring photosynthetic microorganisms include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechococcus sp., Synechocystis sp., Tolypothrix and/or Hematococcus.

A modified Cyanobacteria of the present disclosure may be from any genera or species of Cyanobacteria that is genetically manipulable, i.e., permissible to the introduction and expression of exogenous genetic material. Examples of Cyanobacteria that are known to be engineered according to the methods of the present disclosure include, but are not limited to, the genus Synechocystis, Synechococcus, Thermosynechococcus, Nostoc, Prochlorococcus, Microcystis, Anabaena, Spirulina, and Gloeobacter.

Spirulina are free-floating, filamentous cyanobacteria that include the species Arthrospira platensis and Arthrospira maxima. These two species were formerly classified in the genius Spirulina, but are now classified in the genus Arthrospira. However, the term “Spirulina” remains in use. As used herein “Spirulina” is synonymous with “Arthrospira.” The genus Arthrospria includes 57 species of which 22 are currently taxonomically accepted. Thus, reference to “Spirulina” or “Arthrospira” without further designation includes reference to any of the following species: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

Examples of Cyanobacteria that may be utilized and/or genetically modified according to the methods described herein include, but are not limited to, Chroococcales Cyanobacteria from the genera Aphanocapsa, Aphanothece, Chamaesiphon, Chroococcus, Chroogloeocystis, Coelosphaerium, Crocosphaera, Cyanobacterium, Cyanobium, Cyanodictyon, Cyanosarcina, Cyanothece, Dactylococcopsis, Gloecapsa, Gloeothece, Merismopedia, Microcystis, Radiocystis, Rhabdoderma, Snowella, Synychococcus, Synechocystis, Thermosenechococcus, and Woronichinia; Nostacales Cyanobacteria from the genera Anabaena, Anabaenopsis, Aphanizomenon, Aulosira, Calothrix, Coleodesmium, Cyanospira, Cylindrospermosis, Cylindrospermum, Fremyella, Gleotrichia, Microchaete, Nodularia, Nostoc, Rexia, Richelia, Scytonema, Sprirestis, and Toypothrix; Oscillatoriales Cyanobacteria from the genera Arthrospira, Geitlerinema, Halomicronema, Halospirulina, Katagnymene, Leptolyngbya, Limnothrix, Lyngbya, Microcoleus, Oscillatoria, Phormidium, Planktothricoides, Planktothrix, Plectonema, Pseudoanabaena/Limnothrix, Schizothrix, Spirulina, Symploca, Trichodesmium, Tychonema; Pleurocapsales cyanobacterium from the genera Chroococcidiopsis, Dermocarpa, Dermocarpella, Myxosarcina, Pleurocapsa, Stanieria, Xenococcus; Prochlorophytes Cyanobacterium from the genera Prochloron, Prochlorococcus, Prochlorothrix; and Stigonematales cyanobacterium from the genera Capsosira, Chlorogeoepsis, Fischerella, Hapalosiphon, Mastigocladopsis, Nostochopsis, Stigonema, Symphyonema, Symphonemopsis, Umezakia, and Westiellopsis. In certain embodiments, the Cyanobacterium is from the genus Synechococcus, including, but not limited to Synechococcus bigranulatus, Synechococcus elongatus, Synechococcus leopoliensis, Synechococcus lividus, Synechococcus nidulans, and Synechococcus rubescens.

In certain embodiments, the Cyanobacterium is Anabaena sp. strain PCC 7120, Synechocystis sp. strain PCC6803, Nostoc muscorum, Nostoc ellipsosporum, Nostoc sp. strain PCC 7120, or Synechococcus sp. PCC 7002. In certain embodiments, the Cyanobacterium is S. elongatus sp. strain PCC7942.

Additional examples of Cyanobacteria that may be utilized in the methods provided herein include, but are not limited to, Synechococcus sp. strains WH7803, WH8102, WH8103 (typically genetically modified by conjugation), Baeocyte-forming Chroococcidiopsis spp. (typically modified by conjugation/electroporation), non-heterocyst-forming filamentous strains Planktothrix sp., Plectonema boryanum M101 (typically modified by electroporation), and Heterocyst-forming strains Anabaena sp. strains ATCC 29413 (typically modified by conjugation), Tolypothrix sp. strain PCC 7601 (typically modified by conjugation/electroporation) and Nostoc punctiforme strain ATCC 29133 (typically modified by conjugation/electroporation).

In certain embodiments, the carotenoid accumulation rate of the modified Cyanobacteria is at least about 5-fold greater than the carotenoid accumulation rate of the corresponding wild-type Cyanobacteria. In certain embodiments, the carotenoid accumulation rate of the modified Cyanobacteria is at least about 1.5, 2, 3, 4, 5, 6, 7, 8, 9, 10 or 20-fold greater than a carotenoid accumulation rate of the corresponding wild-type Cyanobacteria. In certain embodiments, the carotenoid accumulation rate is measured at about 24 h, 48 h, 72 h, 96 h, 144 h, and/or 168 h or even greater post-initiation of a stress condition.

In certain embodiments, modified photosynthetic microorganisms, e.g., Cyanobacteria, are grown under conditions favorable for producing carotenoids. In certain embodiments, light intensity is between 100 and 2000 μmoles photons/m²/s, or between 200 and 1000 μmoles photons/m²/s. In certain embodiments, the pH range of culture media is between 6.0 and 10.0. In certain embodiments the pH range of culture media is between 7.0 and 8.5. In certain embodiments, CO₂ is injected into the culture apparatus to a level in the range of 0.05% to 10%. In certain embodiments, the range of CO₂ is between 0.5% and 3%. In certain embodiments, nutrient supplementation is performed during the linear phase of growth. Each of these conditions may be desirable for carotenoid production.

In certain embodiments, the genetically modified, photosynthetic microorganism, e.g., Cyanobacteria, of the present disclosure may be used to produce carotenoids and/or other carbon-containing compounds from just sunlight, water, air, and minimal nutrients, using routine culture techniques of any reasonably desired scale. Among other benefits, the ability to produce large amounts of carotenoids from minimal energy and nutrient input makes the modified photosynthetic microorganism, e.g., Cyanobacteria, of the present disclosure a readily manageable and efficient source of feedstock in the subsequent production of carotenoids.

### Modification of Photosynthetic Microorganisms

In certain embodiments, the present disclosure comprises methods of modifying a photosynthetic microorganism to produce a modified photosynthetic microorganism that produces an increased amount of carotenoids, relative to a corresponding wild-type photosynthetic microorganism or a differently modified photosynthetic microorganism (e.g., one that expresses different carotenoids relative to a corresponding wild-type photosynthetic microorganism), comprising introducing into the microorganism one or more polynucleotides encoding a carotenoid ketolase, carotenoid hydroxylase as described herein, including active fragments or variants thereof.

The methods may further comprise selecting for photosynthetic microorganisms in which the one or more desired polynucleotides were successfully introduced, where the polynucleotides were, e.g., present in a vector that expressed a selectable marker, such as an antibiotic resistance gene. As one example, selection and isolation may include the use of antibiotic resistant markers known in the art (e.g., kanamycin, gentamycin, chloramphenicol, spectinomycin, and streptomycin).

Embodiments of the present disclosure include a cell culture comprising modified Cyanobacteria that have a deletion of a gene in the glycogen synthesis pathway, disruption of a gene in the carotenoid synthesis pathway, up regulation of a gene in the carotenoid synthesis pathway, and/or addition of an exogenous gene encoding an enzyme that functions in the carotenoid synthesis pathway, wherein the modified Cyanobacteria have an increase amount of carotenoid synthesis and accumulation under a stress condition as compared to a corresponding wild-type Cyanobacteria. In certain embodiments, when the modified cyanobacteria grow and/or divide under a condition, such as a nutrient-limited condition, the modified cyanobacteria synthesize and accumulate an increased amount of carotenoids as compared to the corresponding wild-type cyanobacteria. In certain embodiments, wherein the modified Cyanobacteria have addition of an exogenous gene involved in the carotenoid synthesis pathway the modified Cyanobacteria synthesize and accumulate different carotenoids as compared to the corresponding wild-type Cyanobacteria.

Methods of producing a modified photosynthetic microorganism, e.g., a Cyanobacterium, that has an increase carotenoid production as compared to a wild-type photosynthetic microorganism, which may be used in the systems or methods of the present disclosure, include modifying the photosynthetic microorganism so that it has a reduced level of expression of one or more genes of the glycogen-production pathway. In certain embodiments, the one or more genes include glgC, glgA, or a combination thereof. Examples of such glgC genes are provided in SEQ ID NOs:1 (Synechocystis sp. PCC6803), 2 (Synechococcus elongatus PCC7942), 3 (Synechococcus sp. WH8102), 4 (Synechococcus sp. RCC 307), and 5 (Synechococcus sp. PCC 7002), which respectively encode GlgC polypeptides having sequences set forth in SEQ ID NOs: 6, 7, 8, 9, and 10. Examples of such glgA genes are provided in SEQ ID NOs:11 (Synechocystis sp. PCC6803), 12 (Synechococcus elongatus PCC7942), 13 (Synechococcus sp. WH8102), 14 (Synechococcus sp. RCC 307), and 15 (Synechococcus sp. PCC 7002), which respectively encode GlgA polypeptides having sequences set forth in SEQ ID NOs:16, 17, 18, 19 and 20. In certain embodiments, expression or activity is reduced by mutating or deleting a portion or all of the one or more genes. In certain embodiments, expression or activity is reduced by knocking out or knocking down one or more alleles of the one or more genes. Modifications described herein may be produced using standard procedures and reagents, e.g., vectors, readily available and know to those skilled in the art.

In certain embodiments, the method comprises addition of one or more polynucleotides associated with the carotenoid-synthesis pathway of Cyanobacteria to generate the modified Cyanobacteria, wherein the modified cyanobacteria have an increased level of carotenoid synthesis and accumulation as compared to corresponding wild-type Cyanobacteria. In these embodiments and other embodiments, the method comprises transforming Cyanobacteria with a vector containing a polynucleotide sequence of interest and a marker and then isolating the modified Cyanobacteria using the marker.

Photosynthetic microorganisms, e.g., Cyanobacteria may be genetically modified according to techniques known in the art, e.g., to delete a portion or all of a gene or to introduce a polynucleotide that expresses a functional polypeptide. As noted above, in certain aspects, genetic manipulation in photosynthetic microorganisms, e.g., Cyanobacteria, can be performed by the introduction of non-replicating vectors which contain native photosynthetic microorganism sequences, exogenous genes of interest, and selectable markers or drug resistance genes. Upon introduction into the photosynthetic microorganism, the vectors may be integrated into the photosynthetic microorganism's genome through homologous recombination. In this way, an exogenous gene of interest and the drug resistance gene are stably integrated into the photosynthetic microorganism's genome. Such recombinants cells can then be isolated from non-recombinant cells by drug selection. Cell transformation methods and selectable markers for Cyanobacteria are also well known in the art.

Generation of deletions or mutations of any of the one or more genes associated with the glycogen-production pathway and/or carotenoid biosynthesis pathway can be accomplished according to a variety of methods, including those described and exemplified herein. For instance, the instant application describes the use of a non-replicating, selectable vector system that is targeted to the upstream and downstream flanking regions of a given gene (e.g., glgC, glgA, crtG), and which recombines with the Cyanobacterial genome at those flanking regions to replace the endogenous coding sequence with the vector sequence. Given the presence of a selectable marker in the vector sequence, such as a drug selectable marker, Cyanobacterial cells containing the gene deletion can be readily isolated, identified, and characterized. Such selectable vector-based recombination methods need not be limited to targeting upstream and downstream flanking regions, but may also be targeted to internal sequences within a given gene, as long as that gene is rendered “non-functional,” as described herein. As used herein, the term “deletion” includes all techniques for making the gene non-functional even if such techniques do not actually result in removal of any polynucleotides from the genome of a target organism.

Increased expression or overexpression can be achieved a variety of ways, for example, by introducing a polynucleotide into the microorganism, modifying an endogenous gene to overexpress the polypeptide (e.g., by introducing an exogenous regulatory element such as a promoter), or both. For instance, one or more copies of an otherwise endogenous polynucleotide sequence can be introduced by recombinant techniques to increase expression, that is, to create additional copies of the otherwise endogenous polynucleotide sequence. Decreased expression and/or activity can also be achieved a variety of ways, described elsewhere herein and known in the art, including by mutation of coding and/or regulatory sequences of a gene of interest, and/or by RNA inhibition.

In certain aspects, such photosynthetic microorganisms can be further modified by increasing carotenoid hydroxylase activity, for instance, by introducing an exogenous copy of a polynucleotide that encodes a carotenoid hydroxylase, by increasing expression of an endogenous carotenoid hydroxylase, or both. In some aspects, such photosynthetic microorganisms can be further modified by increasing carotenoid ketolase activity, for instance, by introducing an exogenous copy of a polynucleotide that encodes a carotenoid ketolase, by increasing expression of an endogenous carotenoid ketolase protein, or both.

Thus, in certain embodiments, the present disclosure includes methods of producing a modified photosynthetic microorganism, e.g., a Cyanobacteria, comprising: (1) introducing into the photosynthetic microorganism one or more polynucleotides encoding one or more β-carotene hydroxylase proteins and one or more β-carotene ketolase proteins and (2) introducing into the photosynthetic microorganism one or more operatively linked promoters (e.g., inducible or regulable promoters) into a region upstream of the β-carotene hydroxylase and β-carotene ketolase protein coding sequences, and/or introducing one or more polynucleotides encoding a β-carotene hydroxylase or β-carotene ketolase, or a fragment or variant thereof. Exemplary β-carotene hydroxylase proteins include β-carotene 3-hydroxylase and carotenoid 3, 3′-hydroxylase.

Any of the photosynthetic microorganisms described herein can be further modified by reducing expression and/or activity of one or more endogenous genes/proteins associated with glycogen synthesis and/or one or more endogenous genes/proteins associated with carotenoid hydroxylation. Exemplary genes associated with glycogen synthesis and/or storage that may be modified to reduce their expression and/or activity include glgC. Exemplary genes associated with carotenoid biosynthesis that may be modified to reduce their expression and/or activity include crtG.

In certain embodiments, expression or activity is reduced by knocking out or deleting one or more alleles of the one or more genes. Also included is the generation of mutants, such as point mutants, insertions, or full or partial deletions of a gene of interest and/or one or more of its regulatory elements (e.g., promoters, enhancers), to reduce expression and/or activity of a protein of interest. Natural selection or directed selection can also be used to identify naturally-occurring mutants having reduced expression and/or activity of a protein of interest.

Photosynthetic microorganisms, e.g., Cyanobacteria, may be genetically modified according to techniques known in the art, e.g., delete a portion or all of a gene or to introduce a polynucleotide that expresses a functional polypeptide. As noted above, in certain aspects, genetic manipulation in photosynthetic microorganisms, e.g., Cyanobacteria, can be performed by the introduction of non-replicating vectors which contain native photosynthetic microorganism sequences, exogenous genes of interest, and selectable markers or drug resistance genes. Upon introduction into the photosynthetic microorganism, the vectors may be integrated into the photosynthetic microorganism's genome through homologous recombination. In this way, an exogenous gene of interest and the drug resistance gene are stably integrated into the photosynthetic microorganism's genome. Such recombinant cells can then be isolated from non-recombinant cells by drug selection. Cell transformation methods and selectable markers for Cyanobacteria are also well known in the art.

Techniques for producing such alterations or changes, such as by recombination with a vector having a selectable marker, are exemplified herein and known in the molecular biological art. In certain embodiments, one or more alleles of a gene, e.g., two or all alleles, may be mutated or deleted within a photosynthetic microorganism. In certain embodiments, modified photosynthetic microorganisms, e.g., Cyanobacteria, of the present disclosure are merodiploids or partial diploids.

### Modifications to Glycogen Biosynthesis

Glycogen is a polysaccharide of glucose, which functions as a means of carbon and energy storage in most cells, including animal and bacterial cells. More specifically, glycogen is a very large branched glucose homopolymer containing about 90% α-1,4-glucosidic linkages and 10% α-1,6 linkages. For bacteria in particular, the biosynthesis and storage of glycogen in the form of α-1,4-polyglucans represents an important strategy to cope with transient starvation conditions in the environment.

Glycogen biosynthesis involves the action of several enzymes. For instance, bacterial glycogen biosynthesis occurs generally through the following general steps: (1) formation of glucose-1-phosphate, catalyzed by phosphoglucomutase (Pgm), followed by (2) ADP-glucose synthesis from ATP and glucose 1-phosphate, catalyzed by glucose-1-phosphate adenylyltransferase (GlgC), followed by (3) transfer of the glucosyl moiety from ADP-glucose to a pre-existing α-1,4 glucan primer, catalyzed by glycogen synthase (GlgA). This latter step of glycogen synthesis typically occurs by utilizing ADP-glucose as the glucosyl donor for elongation of the α-1,4-glucosidic chain.

In bacteria, the main regulatory step in glycogen synthesis takes place at the level of ADP-glucose synthesis, or step (2) above, the reaction catalyzed by glucose-1-phosphate adenylyltransferase (GlgC), also known as ADP-glucose pyrophosphorylase. In contrast, the main regulatory step in mammalian glycogen synthesis occurs at the level of glycogen synthase (e.g., GlgA). As shown herein, by altering the regulatory and/or other active components in the glycogen synthesis pathway of photosynthetic microorganisms such as Cyanobacteria, and thereby reducing the biosynthesis and storage of glycogen, the carbon that would have otherwise been stored as glycogen can be utilized by the photosynthetic microorganism to synthesize other carbon-based molecules, such as carotenoids particularly under stress conditions such as nutrient deficiency.

Therefore, certain modified photosynthetic microorganisms, e.g., Cyanobacteria, of the present disclosure may comprise a mutation, deletion, or any other alteration that disrupts one or more of these steps (i.e., renders the one or more steps “non-functional” with respect to glycogen biosynthesis and/or storage), or alters any one or more of the enzymes directly involved in these steps, or the genes encoding them.

In certain embodiments, a modified photosynthetic microorganism, e.g., a Cyanobacterium, expresses a reduced amount of a glucose-1-phosphate adenylyltransferase (glgC) gene. In certain embodiments, it may comprise a mutation or deletion in the glgC gene, including any of its regulatory elements. The enzyme encoded by the glgC gene (e.g., glucose-1-phosphate adenylyl transferase) participates generally in starch, glycogen and sucrose metabolism by catalyzing the following chemical reaction:

ATP+alpha-D-glucose1-phosphate⇄diphosphate+ADP-glucose

Thus, the two substrates of this enzyme are ATP and alpha-D-glucose 1-phosphate, whereas its two products are diphosphate and ADP-glucose. The GlgC-encoded enzyme catalyzes the first committed and rate-limiting step in starch biosynthesis in plants and glycogen biosynthesis in bacteria. It is the enzymatic site for regulation of storage polysaccharide accumulation in plants and bacteria, being allosterically activated or inhibited by metabolites of energy flux.

The enzyme encoded by the glgC gene belongs to a family of transferases, specifically those transferases that transfer phosphorus-containing nucleotide groups (i.e., nucleotidyl-transferases). The systematic name of this enzyme class is typically referred to as ATP:alpha-D-glucose-1-phosphate adenylyltransferase. Other names in common use include ADP glucose pyrophosphorylase, glucose 1-phosphate adenylyltransferase, adenosine diphosphate glucose pyrophosphorylase, adenosine diphosphoglucose pyrophosphorylase, ADP-glucose pyrophosphorylase, ADP-glucose synthase, ADP-glucose synthetase, ADPG pyrophosphorylase, and ADP:alpha-D-glucose-1-phosphate adenylyltransferase.

The glgC gene is expressed in a wide variety of plants and bacteria, including most, if not all, Cyanobacteria. The glgC gene is also fairly conserved among Cyanobacteria.

In certain embodiments, a modified photosynthetic microorganism comprises modifications, such that it has reduced expression of one or more genes associated with a glycogen synthesis or storage pathway and/or increased expression of one or more polynucleotides that encode a protein associated with a glycogen breakdown pathway, or a functional variant of fragment thereof.

In various embodiments, modified photosynthetic microorganisms, e.g., Cyanobacteria, of the present disclosure have reduced expression of one or more genes associated with glycogen synthesis and/or storage. In certain embodiments, these modified photosynthetic microorganisms have a mutated, deleted, or otherwise non-functional gene associated with glycogen synthesis and/or storage. In certain embodiments, these modified photosynthetic microorganisms comprise a vector that includes a portion of a mutated or deleted gene, e.g., a targeting vector used to generate a knockout or knockdown of one or more alleles of the mutated or deleted gene. Deletion of the glgC gene in Cyanobacteria, such as Synechococcus, reduces the accumulation of glycogen in the Cyanobacteria, and increases the production of other carbon-based products, such as carotenoids.

In certain embodiments, an introduced promoter is used to regulate transcription of a glycogen pathway gene. In certain embodiments, the introduced promoter is exogenous or foreign to the photosynthetic microorganism, i.e., it is derived from a genus/species that differs from the microorganism being modified. In other embodiments, the introduced promoter is a recombinantly introduced copy of an otherwise endogenous or naturally-occurring promoter sequence, i.e., it is derived from the same genus or species of microorganism being modified.

In certain embodiments, the one or more introduced promoters are present in one or more expression constructs. In certain embodiments, the one or more introduced promoters comprise one or more inducible promoters. An inducible promoter can be introduced typically upstream of the microorganism's natural coding region for a gene of interest and/or the inducible promoter can be encoded by an introduced polynucleotide (e.g., vector) that encodes the promoter and the gene of interest. In certain embodiments, the one or more expression constructs are stably integrated into the genome of the modified photosynthetic microorganism. In certain embodiments, the introduced polynucleotide encoding an introduced protein is present in an expression construct comprising a weak promoter under non-induced conditions. In certain embodiments, one or more of the introduced polynucleotides are codon-optimized for expression in a Cyanobacterium, e.g., a Synechococcus elongatus.

In certain embodiments, the modified photosynthetic microorganisms are cultured under conditions that lack a regulator capable of inducing the inducible promoter. In an embodiment the regulator is a nutrient. In an embodiment the regulator is nitrogen. In an embodiment the nirA promoter that is induced by nitrite may be used as the inducible promoter when the regulator is nitrogen. Thus, deprivation of the nutrient creates a stress condition and also ceases induction of the inducible promoter. Conditions and reagents suitable for inducing inducible promoters are known and available in the art. The inducible promoter may, in an embodiment, control transcription of glycogen pathway gene (e.g., glgC). Endogenous copies of the same or similar glycogen pathway gene may be rendered non-functional such that all transcription of the gene of interest is regulated by the inducible promoter. Thus a given glycogen pathway gene may be transcribed, or transcribed at wild-type levels, by the modified photosynthetic microorganism only when the nutrient is present. Absence of the nutrient therefore creates a stress condition and reduces or stops transcription of the glycogen pathway gene.

### Modifications to Carotenoid Biosynthesis

In certain aspects, the modified photosynthetic organisms described herein are further modified to increase production of carotenoids, for instance, by introducing and/or overexpressing one or more polypeptides associated with carotenoid synthesis. Examples of carotenoids that may be increased include xanthophylls such as zeaxanthin, astaxanthin, and canthaxanthin.

In certain embodiments, a modified photosynthetic microorganism comprises an introduced polynucleotide that encodes one or more carotenoid biosynthesis proteins. In some instances, a modified photosynthetic microorganism comprises an endogenous polynucleotide that encodes a carotenoid biosynthesis protein, where a regulatory element such as an inducible or non-inducible promoter is introduced upstream of that polynucleotide to regulate or alter expression of the encoded protein.

Embodiments of the present disclosure include polynucleotides encoding β-carotene oxygenase, β-carotene hydroxylase, canthaxanthin hydroxylase (i.e., a carotenoid 3,3′-hydroxylase that recognizes canthaxanthin), or β-carotene ketolase. Such polynucleotides can be partially or fully isolated from other cellular components, within a vector, for example, a composition comprising such a vector (e.g., in a tube or kit), or in a host cell, such as modified photosynthetic microorganism.

Also included are nucleotide sequences that encode any functional naturally-occurring variants or fragments (e.g., allelic variants, orthologs, splice variants) or non-naturally occurring variants or fragments of these native polynucleotides (i.e., optimized by engineering), as well as compositions comprising such polynucleotides, including, for example, cloning and expression vectors.

In certain embodiments, a modified photosynthetic microorganism comprises reduced or eliminated expression or activity of a carotenoid biosynthesis polypeptide. Included are full or partial deletions, and point mutations or insertions into an endogenous carotenoid biosynthesis gene that reduce or eliminate expression and/or activity of the encoded polypeptide.

Increased expression can be achieved a variety of ways, for example, by introducing a polynucleotide into the photosynthetic organism, modifying an endogenous gene to overexpress the corresponding polypeptide, or both. For instance, one or more copies of an otherwise endogenous polynucleotide sequence can be introduced by recombinant techniques to increase expression, and/or a promoter/enhancer sequence can be introduced upstream of an endogenous gene to up-regulate expression.

Certain embodiments thus include modified photosynthetic microorganisms that accumulate an increased amount of carotenoids as compared to the wild-type photosynthetic microorganism, and which comprise one or more introduced polynucleotides that encode one or more enzymes having carotenoid hydroxylase or carotenoid ketolase activity. Optionally, to further increase production of carotenoids, such photosynthetic microorganisms can further comprise one or more introduced or overexpressed polynucleotides that encode a carotenoid hydroxylase, carotenoid ketolase, or any combination thereof.

The principles described herein can apply to an introduced polynucleotide which encodes a carotenoid hydroxylase (e.g., β-carotene hydroxylase) or other overexpressed polypeptide. For instance, in certain embodiments, the introduced polynucleotide encoding the carotenoid hydroxylase or other polypeptide is exogenous or foreign to the photosynthetic microorganism, i.e., it is derived from a genus/species that differs from the microorganism being modified. In other embodiments, the introduced polynucleotide is a recombinantly introduced copy of an otherwise endogenous or naturally-occurring sequence, i.e., it is derived from the same species of microorganism being modified.

For example, to produce carotenoids, a modified photosynthetic microorganism may comprise an overexpressed carotenoid hydroxylase (e.g., β-carotene hydroxylase). In these and related embodiments, carotenoid production can be further increased by subjecting the modified photosynthetic microorganism to a stress condition. One illustrative carotenoid hydroxylase is encoded by crtR of Synechococcus elongatus PCC7942 (SEQ ID NO: 21). Another illustrative carotenoid hydroxylase is encoded by crtZ of Pantoea ananatis (SEQ ID NO: 22). Also included are homologs or paralogs thereof, functional equivalents thereof, and fragments or variants thereofs. Functional equivalents can include carotenoid hydroxylase with the ability to add hydroxyl groups to β-carotene. These and related embodiments can be further combined with reduced expression and/or activity of an endogenous gylcogen-pathway gene (e.g., glgC in S. elongatus), described herein, to shunt carbon away from glycogen production and towards carotenoids.

In certain embodiments, the exogenous nucleic acid does not comprise a nucleic acid sequence that is native to the microorganism's genome. In some embodiments, the exogenous nucleic acid comprises a nucleic acid sequence that is native to the microorganism's genome, but it has been introduced into the microorganism, e.g., in a vector or by molecular biology techniques, for example, to increase expression of the nucleic acid and/or its encoded polypeptide in the microorganism. In certain embodiments, the expression of a native or endogenous nucleic acid and its corresponding protein can be increased by introducing a heterologous promoter upstream of the native gene.

### Polynucleotide Variants, Fragments, Vectors, and Expression Systems

The present invention contemplates the use in the methods described herein of variants of full-length enzymes having, carotenoid hydroxylase activity, carotenoid ketolase activity, β-carotene hydroxylase activity, and/or β-carotene oxygenase activity, truncated fragments of these full-length polypeptides, variants of truncated fragments, as well as their related biologically active fragments. Typically, biologically active fragments of a polypeptide may participate in an interaction, for example, an intra-molecular or an inter-molecular interaction. An inter-molecular interaction can be a specific binding interaction or an enzymatic interaction (e.g., the interaction can be transient and a covalent bond is formed or broken).

The disclosure also describes variants of polynucleotide sequences. Nucleic acid variants can be naturally-occurring, such as allelic variants (same locus), homologs (different locus), and orthologs (different organism) or can be non-naturally-occurring. Naturally occurring variants such as these can be identified and isolated using well-known molecular biology techniques including, for example, various polymerase chain reaction (PCR) and hybridization-based techniques as known in the art. Naturally occurring variants can be isolated from any organism that encodes one or more genes having an activity of a reference polypeptide. Embodiments of the present invention, therefore, encompass Cyanobacteria comprising such naturally-occurring polynucleotide variants.

Non-naturally occurring variants can be made by mutagenesis techniques, including those applied to polynucleotides, cells, or organisms. The variants can contain nucleotide substitutions, deletions, inversions, and insertions. Variation can occur in either or both the coding and non-coding regions. In certain aspects, non-naturally occurring variants may have been optimized for use in Cyanobacteria, such as by engineering and screening the enzymes for increased activity, stability, or any other desirable feature.

The variations can produce both conservative and non-conservative amino acid substitutions (as compared to the originally encoded product). For nucleotide sequences, conservative variants include those sequences that, because of the degeneracy of the genetic code, encode the amino acid sequence of a reference polypeptide. Variant nucleotide sequences also include synthetically derived polynucleotide sequences, such as those generated, for example, by using site-directed mutagenesis but which still encode a biologically active reference polypeptide, as described elsewhere herein. Generally, variants of a particular polynucleotide sequence will have at least about 30%, 40% 50%, 55%, 60%, 65%, 70%, generally at least about 75%, 80%, 85%, 90%, 95%, or 98% or more sequence identity to a reference polynucleotide sequence as determined by sequence alignment programs described elsewhere herein using default parameters.

Known reference polynucleotide sequences (e.g., wild-type sequences previously characterized) can be used to isolate corresponding sequences and alleles from other organisms, particularly other microorganisms. Methods are readily available in the art for the hybridization of nucleic acid sequences. Coding sequences from other organisms may be isolated according to well-known techniques based on their sequence identity with the coding sequences set forth herein. In these techniques all or part of the known coding sequence is used as a probe which selectively hybridizes to other reference coding sequences present in a population of cloned genomic DNA fragments or cDNA fragments (i.e., genomic or cDNA libraries) from a chosen organism.

Accordingly, the present disclosure also includes polynucleotides that hybridize to reference nucleotide sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used.

Reference herein to “low stringency” conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at room temperature. One embodiment of low stringency conditions includes hybridization in 6×sodium chloride/sodium citrate (SSC) at about 45° C., followed by two washes in 0.2×SSC, 0.1% SDS at least at 50° C. (the temperature of the washes can be increased to 55° C. for low stringency conditions).

“Medium stringency” conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO4 (pH 7.2), 7% SDS for hybridization at 65° C., and (i) 2×SSC, 0.1% SDS; or (ii) 0.5% BSA, 1 mM EDTA, 40 mM NaHPO4 (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

“High stringency” conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO4 (pH 7.2), 7% SDS for hybridization at 65° C., and (i) 0.2×SSC, 0.1% SDS; or (ii) 0.5% BSA, 1 mM EDTA, 40 mM NaHPO4 (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a reference polypeptide or enzyme described herein is encoded by a polynucleotide that hybridizes to a disclosed nucleotide sequence under very high stringency conditions. One embodiment of very high stringency conditions includes hybridizing in 0.5 M sodium phosphate, 7% SDS at 65° C., followed by one or more washes in 0.2×SSC, 1% SDS at 65° C.

Other stringency conditions are well known in the art and the skilled artisan will recognize that various factors can be manipulated to optimize the specificity of the hybridization. Optimization of the stringency of the final washes can serve to ensure a high degree of hybridization.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tm for formation of a DNA-DNA hybrid. It is well known in the art that the Tm is the melting temperature, or temperature at which two complementary polynucleotide sequences dissociate. Methods for estimating Tₘ are well known in the art.

In general, the Tm of a perfectly matched duplex of DNA may be predicted as an approximation by the formula: Tm=81.5+16.6 (log₁₀ M)+0.41 (% G+C)−0.63 (% formamide)−(600/length) wherein: M is the concentration of Na+, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In an example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5× Reinhardt's solution (0.1% ficoll™, 0.1% polyvinylpyrollidone and 0.1% bovine serum albumin), 0.1% SDS and 200 mg/mL denatured salmon sperm DNA) containing a labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.).

Polynucleotides and fusions thereof may be prepared, manipulated, and/or expressed using any of a variety of well-established techniques known and available in the art. For example, polynucleotide sequences which encode polypeptides of the invention, or fusion proteins or functional equivalents thereof, may be used in recombinant DNA molecules to direct expression of a carotenoid biosynthesis enzyme in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. Such nucleotides are typically referred to as “codon-optimized.”

Moreover, the polynucleotide sequences described herein can be engineered using methods generally known in the art in order to alter polypeptide encoding sequences for a variety of reasons, including but not limited to, alterations which modify the cloning, processing, expression and/or activity of the gene product.

In order to express a desired polypeptide, a nucleotide sequence encoding the polypeptide, or a functional equivalent, may be inserted into appropriate expression vector, i.e., a vector that contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination.

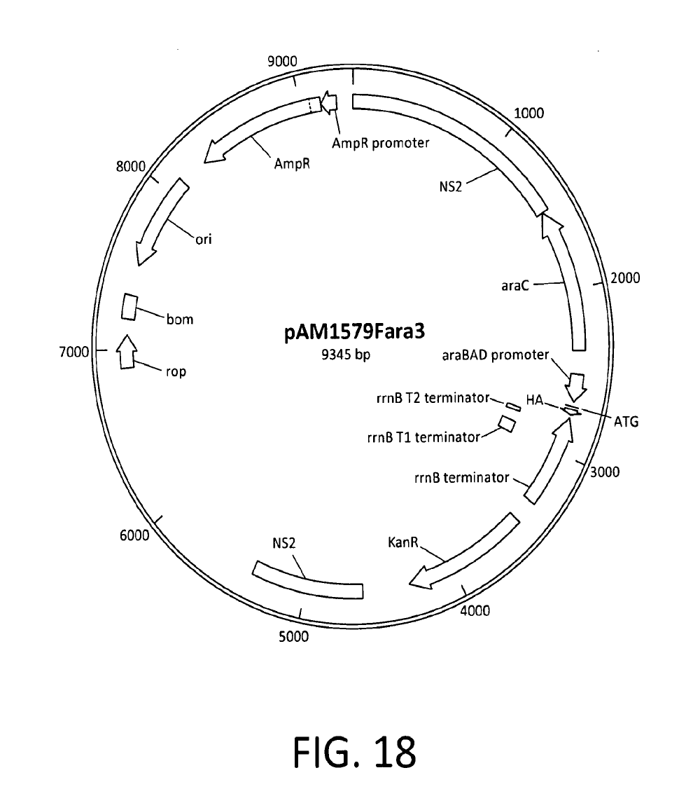

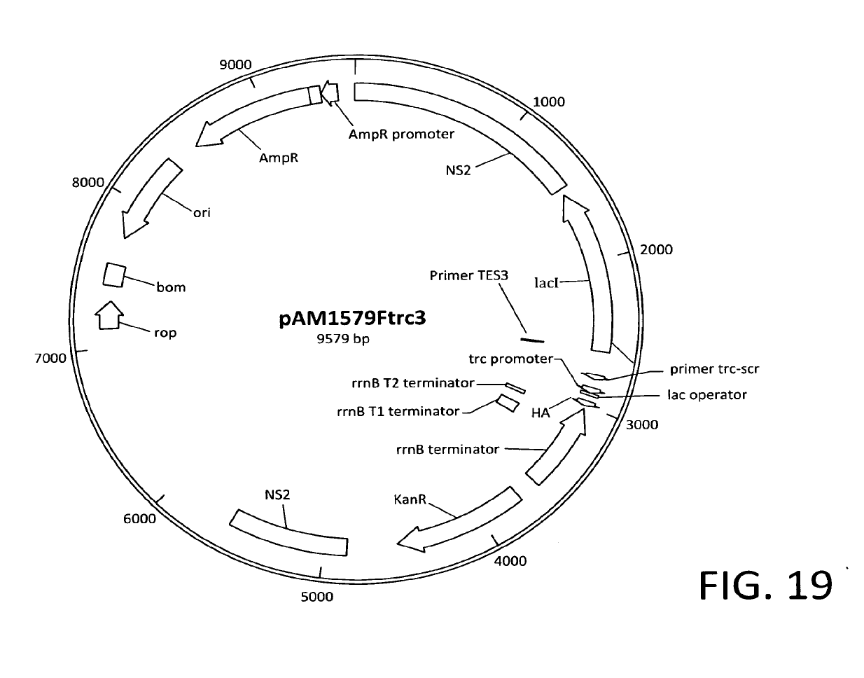

A variety of expression vector/host systems are known and may be utilized to contain and express polynucleotide sequences. In certain embodiments, the polynucleotides of the present disclosure may be introduced and expressed in Cyanobacterial systems. As such, the present disclosure contemplates the use of vector and plasmid systems having regulatory sequences (e.g., promoters and enhancers) that are suitable for use in various Cyanobacteria. In an example the pTJ001/pMX570 plasmid (SEQ ID NO: 23) that include a pTrc promoter and genes for spectinomycin and streptomycin may be used. In an example, the pAM1579Fara3 plasmid (SEQ ID NO: 24) that includes a pBAD promoter and a gene for kanamycin resistance may be used. In an example the pAM1579Ftrc3 plasmid (SEQ ID NO: 25) that includes a pTrc promoter and gene for kanamycin resistance may be used. FIG. 18 shows the pAM1579Fara3 plasmid. FIG. 19 shows the pAM1579Ftrc3 plasmid.

The “control elements” or “regulatory sequences” present in an expression vector (or employed separately) are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. Generally, it is well-known that strong E. coli promoters work well in Cyanobacteria. Also, when cloning in Cyanobacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. Other vectors containing IPTG inducible promoters, such as pAM1579 and pAM2991trc, may be utilized according to the present invention.

In Cyanobacterial systems, a number of expression vectors or regulatory sequences may be selected depending upon the use intended for the expressed polypeptide. When large quantities are needed, vectors or regulatory sequences which direct high level expression of encoded proteins may be used. For example, overexpression of β-carotene hydroxylase may be utilized to increase biosynthesis of zeaxanthin. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-carotene hydroxylase so that a hybrid protein is produced; pIN vectors; and the like.

Certain embodiments may employ Cyanobacterial promoters or regulatory operons. In certain embodiments, a promoter may comprise an rbcLS operon of Synechococcus, or a cpc operon of Synechocystis sp. strain PCC 6714. In certain embodiments, the tRNApro gene from Synechococcus may also be utilized as a promoter. Certain embodiments may employ the nirA promoter from Synechococcus sp. strain PCC7942, which is repressed by ammonium and induced by nitrite. The efficiency of expression may be increased by the inclusion of enhancers which are appropriate for the particular Cyanobacterial cell system which is used, such as those described in the literature.

In certain embodiments, expression vectors or introduced promoters utilized to overexpress an exogenous or endogenous reference polypeptide, or fragment or variant thereof, comprise a weak promoter under non-inducible conditions, e.g., to avoid toxic effects of long-term overexpression of any of these polypeptides. One example of such a vector for use in Cyanobacteria is the pBAD vector system. Expression levels from any given promoter may be determined, e.g., by performing reverse transcription and quantitative polymerase chain reaction (RT-qPCR) to determine the amount of transcript or mRNA produced by a promoter, e.g., before and after induction. In certain instances, a weak promoter is defined as a promoter that has a basal level of expression of a gene or transcript of interest, in the absence of inducer, that is <2.0% of the expression level produced by the promoter of the rnpB gene in S. elongatus PCC7942. In other embodiments, a weak promoter is defined as a promoter that has a basal level of expression of a gene or transcript of interest, in the absence of inducer, that is <5.0% of the expression level produced by the promoter of the mpB gene in S. elongatus PCC7942.

It will be apparent that further to their use in vectors, any of the regulatory elements described herein (e.g., promoters, enhancers, repressors, ribosome binding sites, transcription termination sites) may be introduced directly into the genome of a photosynthetic microorganism (e.g., Cyanobacterium), typically in a region surrounding (e.g., upstream or downstream of) an endogenous or naturally-occurring reference gene/polynucleotide sequence described herein, to regulate expression (e.g., facilitate overexpression) of that gene.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). The presence or expression levels of a desired polynucleotide may also be confirmed by PCR.

### Culturing of Photosynthetic Microorganisms

The modified photosynthetic microorganisms of the present disclosure may be cultured under stress conditions to increase production of carotenoids. Accordingly, the present disclosure provides methods of culturing carotenoids, comprising culturing any of the modified photosynthetic microorganisms of the present disclosure (described elsewhere herein) under conditions that include stress conditions. In an embodiment, the modified photosynthetic microorganism is a Cyanobacterium that produces or accumulates increased carotenoids relative to an unmodified or wild-type Cyanobacterium of the same species, or a differently modified Cyanobacterium of the same species.

In certain embodiments, the one or more introduced polynucleotides are present in one or more expression constructs. In certain embodiments, the one or more expression constructs comprises one or more inducible promoters (e.g., pTrc). In certain embodiments, the one or more expression constructs are stably integrated into the genome of the modified photosynthetic microorganism. In certain embodiments the inducible promoters are induced by presence of a nutrient, signal molecule, or environmental condition. When the nutrient, signal molecule, or environmental condition is present, the photosynthetic microorganism will produce the gene product regulated by the inducible promoter.

In certain embodiments, the introduced polynucleotide encoding an introduced protein is present in an expression construct comprising a weak promoter under non-induced conditions. In certain embodiments, one or more of the introduced polynucleotides are codon-optimized for expression in a Cyanobacterium, e.g., a Synechococcus elongatus. Thus, when the promoter is induced by a nutrient, absence of that nutrient results in only weak expression of the corresponding gene product.

Photosynthetic microorganisms may be cultured according to techniques known in the art. For example, Cyanobacteria may be cultured or cultivated according to techniques known in the art including growth in a photobioreactor. One example of typical laboratory culture conditions for Cyanobacterium is growth in BG-11 medium (ATCC Medium 616) at 30° C. in a vented culture flask with constant agitation and constant illumination at 30-100 μmole photons m⁻² s⁻¹.

In certain embodiments, modified photosynthetic microorganisms, e.g., Cyanobacteria, are grown under stress conditions. The stress conditions may include high light, high salt, or nutrient deprivation such as nitrogen deprivation, sulfur deprivation, phosphorous deprivation, and/or iron deprivation. In certain embodiments, high light is achieved by light intensity between 200 and 1000 μmoles photons m⁻² s⁻¹, or between 200 and 300 μmoles photons m⁻² s⁻¹. In certain embodiments, the pH range of culture media is between 7.0 and 10.0. In certain embodiments, CO₂ is injected into the culture apparatus to a level in the range of 1% to 10%. In certain embodiments, the range of CO₂ is between 0.05% and 0.1%. In certain embodiments, nutrient deprivation is performed during the linear phase of growth. In certain embodiments the nutrient deprivation is applied after a brief washing phase that removes or reduces the nutrient in question.

A wide variety of mediums are available for culturing Cyanobacteria, including, for example, Aiba and Ogawa (AO) Medium, Allen and Arnon Medium plus Nitrate (ATCC Medium 1142), Antia's (ANT) Medium, Aquil Medium, Ashbey's Nitrogen-free Agar, ASN-III Medium, ASP 2 Medium, ASW Medium (Artificial Seawater and derivatives), ATCC Medium 617 (BG-11 for Marine Blue-Green Algae; Modified ATCC Medium 616 [BG-11 medium]), ATCC Medium 819 (Blue-green Nitrogen-fixing Medium; ATCC Medium 616 [BG-11 medium] without NO3), ATCC Medium 854 (ATCC Medium 616 [BG-11 medium] with Vitamin B12), ATCC Medium 1047 (ATCC Medium 957 [MN marine medium] with Vitamin B12), ATCC Medium 1077 (Nitrogen-fixing marine medium; ATCC Medium 957 [MN marine medium] without NO3), ATCC Medium 1234 (BG-11 Uracil medium; ATCC Medium 616 [BG-11 medium] with uracil), Beggiatoa Medium (ATCC Medium 138), Beggiatoa Medium 2 (ATCC Medium 1193), BG-11 Medium for Blue Green Algae (ATCC Medium 616), Blue-Green (BG) Medium, Bold's Basal (BB) Medium, Castenholtz D Medium, Castenholtz D Medium Modified (Halophilic cyanobacteria), Castenholtz DG Medium, Castenholtz DGN Medium, Castenholtz ND Medium, Chloroflexus Broth, Chloroflexus Medium (ATCC Medium 920), Chu's #10 Medium (ATCC Medium 341), Chu's #10 Medium Modified, Chu's #11 Medium Modified, DCM Medium, DYIV Medium, E27 Medium, E31 Medium and Derivatives, f/2 Medium, f/2 Medium Derivatives, Fraquil Medium (Freshwater Trace Metal-Buffered Medium), Gorham's Medium for Algae (ATCC Medium 625), h/2 Medium, Jaworski's (JM) Medium, K Medium, L1 Medium and Derivatives, MN Marine Medium (ATCC Medium 957), Plymouth Erdschreiber (PE) Medium, Prochlorococcus PC Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Spirulina: ATCC Medium 1679, Spirulina (SP) Medium, van Rijn and Cohen (RC) Medium, Walsby's Medium, Yopp Medium, and Z8 Medium, among others.

### EXAMPLES

Certain embodiments of the present disclosure now will be illustrated by the following Examples. The present disclosure may, however, be embodied in many different forms and should not be construed as limited to the embodiments set forth herein; rather, these embodiments are provided so that this disclosure will be thorough and complete, and will fully convey the scope of the disclosure to those skilled in the art.

### Example 1—Disruption of the Carotenoid Biosynthesis Pathway

A mutant S. elongatus, ΔcrtG, was obtained from a mutant library. The ΔcrtG mutant has a transposon insertion into the 2,2′β-hydroxylase (crtG) gene of a wild-type S. elongatus inactivating the crtG gene (SEQ ID NO: 26). This technique for inactivating the crtG gene may be referred to as “interrupting” the crtG gene and is annotated by ΔcrtG. The transposon includes a kanamycin resistance marker (SEQ ID NO: 27) that allows for selection. A carotenoid synthesis pathway shown in FIG. 1 in Synechococcus converts β-carotene to zeaxanthin with β-carotene hydroxylase (crtR) then converts zeaxanthin to caloxanthin with crtG and subsequently converts caloxanthin to nostoxanthin also with crtG. Effects of disrupting this gene in the carotenoid biosynthesis pathway of S. elongatus were not previously known.

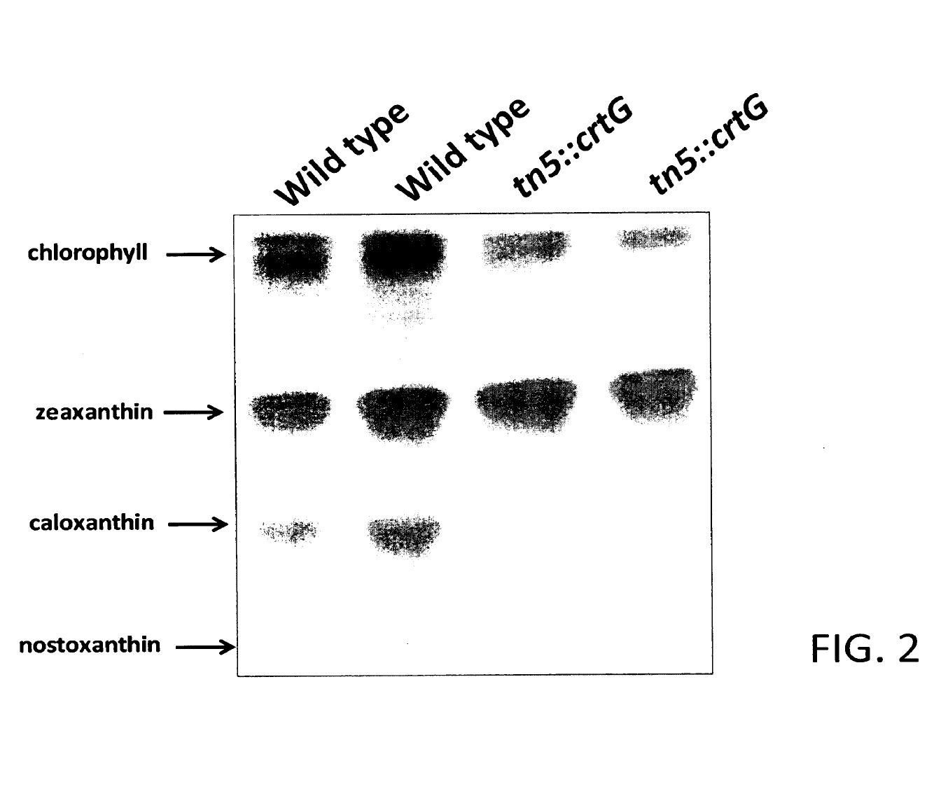

FIG. 2 shows a thin-layer chromatography (TLC) gel of carotenoids extracted from ΔcrtG. This ΔcrtG S. elongatus was grown in BG-11 media at 100 μmoles photons m⁻² s⁻¹ for 48 hr. Once the optical density of the culture at 750 nm reached 1 absorbance unit, 1 mL of cultured was centrifuged at 3500×g for 10 min. Carotenoids were extracted from the resulting cell pellet with 1 mL of acetone. The acetone in the pigment extract was evaporated to dryness, and the dried pigments were resuspended in 20 μL acetone. These pigments were spotted onto a silica TLC plate and developed with a solution of diethyl ether/hexane/methanol/acetic acid (60:40:5:1). The TLC results indicate that changing the wild-type pathway by deleting the crtG gene greatly reduces synthesis of downstream products (i.e., caloxanthin and nostoxanthin) and increases synthesis of zeaxanthin. Surprisingly, the cells remained viable following deletion of crtG.

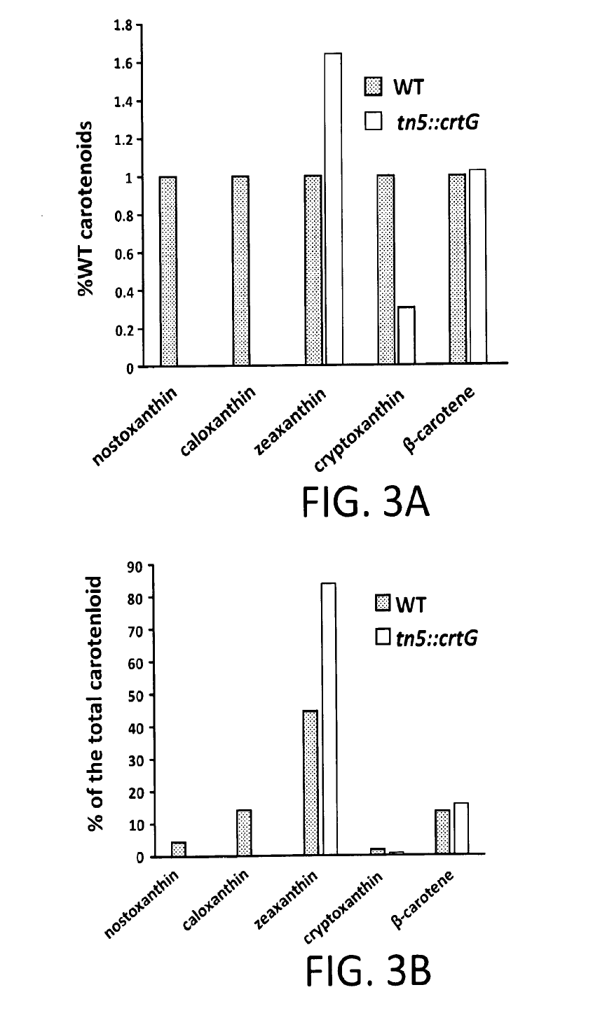

FIG. 3A shows the results of LC-MS analysis of percent various carotenoids were of total carotenoid dry-weight in wild-type S. elongatus and ΔcrtG S. elongatus. LC-MS analysis was done performed on a Waters 2695 instrument equipped with diode array (Waters 2998) and mass spectrometer (Waters micromass ZQ). The separation was performed on a C8 column with two solutions; 0.1% trifluoroacetic acid (in H₂O, solution A) and 1:1 acetonitrile/methanol with 0.1% trifluoroacetic acid (solution B). After injection of the sample onto the column, the analysis takes 25 minutes and can be broken into three steps. In step 1, an 80/20 mixture of A/B is reversed to a 20/80 mixture of A/B by a linear gradient over the span of 3 minutes. In step 2, the 20/80 mixture of A/B is altered to 100% B over the span of 17 minutes (linear gradient). Finally the column is washed with 100% B for 5 minutes. The carotenoid content in ΔcrtG is relative to wild-type S. elongatus. Zeaxanthin increased to about 1.6× the wild-type level to about 83% of the total carotenoids in ΔcrtG, β-carotene levels were largely unchanged, while the levels of caloxanthin, nostoxanthin, and cryptoxanthin decreased greatly or were entirely undetected. FIG. 3B shows changes to the relative percent of total carotenoids by dry weight after the crtG interruption. The amount of zeaxanthin increased from about 47% of total carotenoids in the wild-type to about 83% of the total carotenoids in ΔcrtG.

### Example 2—Nitrogen Deprivation

The glycogen pathway in Synechococcus converts other carbon-containing molecules generated by the cell into glycogen. Disruption of the glycogen pathway causes Synechococcus to process carbon-containing molecules differently. A strain of S. elongates, ΔglgC, was created by knocking out the glgC gene (SEQ ID NO: 2; which encodes glucose-1-phosphate adenylyltransferase) with addition of a gentamycin resistance cartridge (SEQ ID NO: 28). Gentamycin resistance was used to screen for successful knockouts that lacked a functional glgC gene.

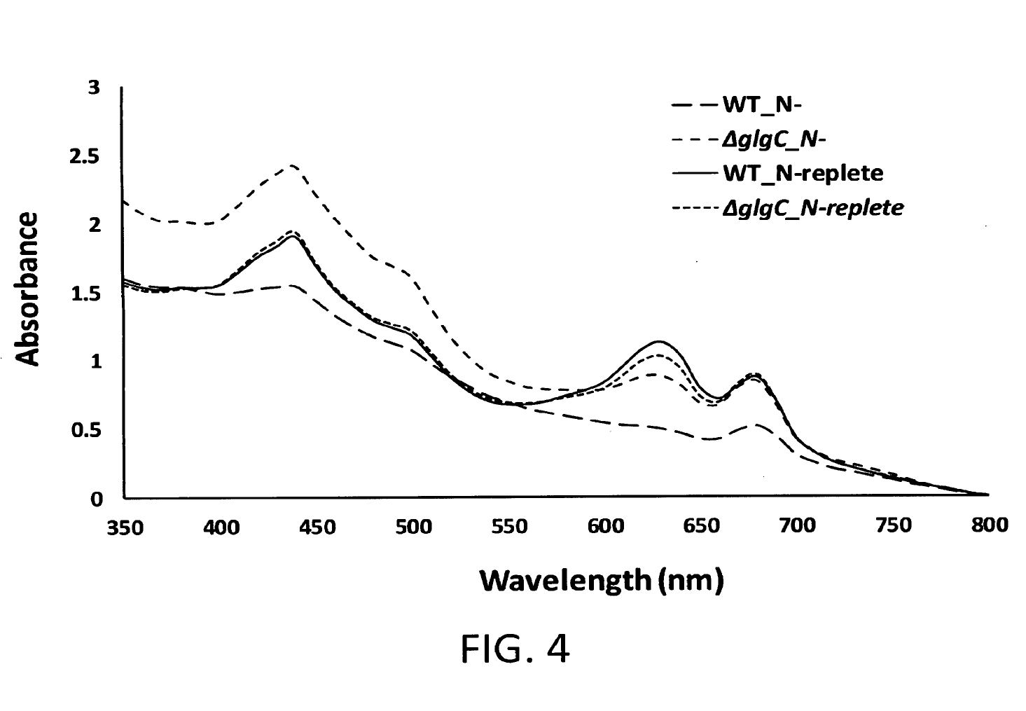

Both the wild-type strain and the ΔglgC strain were exposed to nitrogen replete conditions and nitrogen starvation. Nitrogen starvation was achieved by spinning cells out of their nitrogen replete growth media (BG-11 media including 17 mM NaNO₃) and resuspending them in BG-11 media without NaNO₃ (the cells were washed once before resuspension). FIG. 4 shows changes in light absorption. A spectramax M5 was used to measure the absorption of light between 350 nm and 800 nm. The ΔglgC strain under nitrogen starvation exhibited a dramatic increase in absorbance at 440 nm suggesting increased levels of carotenoids. This dramatic increase is shown only with the combination of glgC deletion and nitrogen starvation.

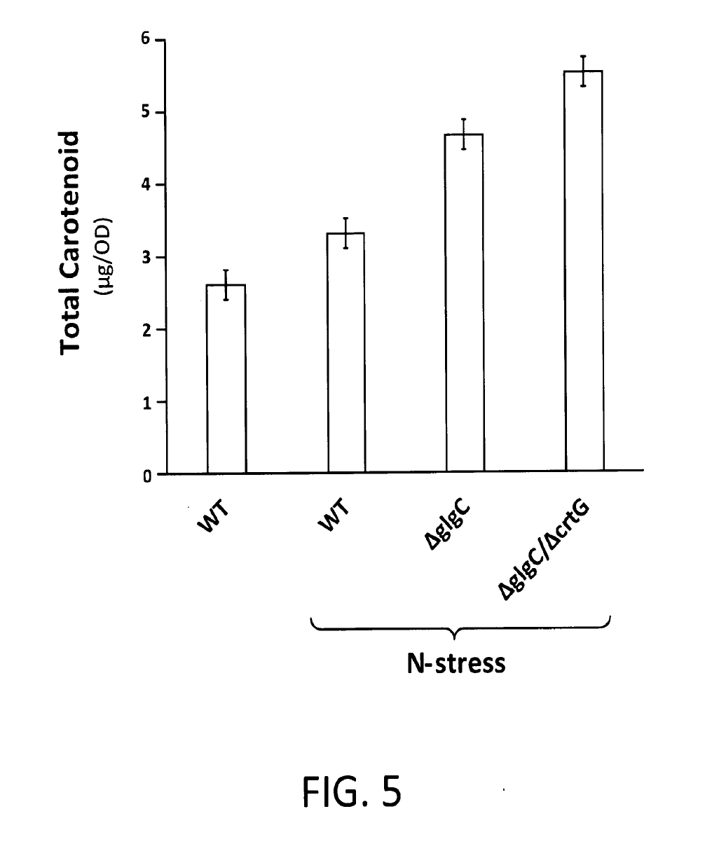

FIG. 5 shows levels of total carotenoids present in wild-type Synechococcus under replete conditions in comparison to levels of total carotenoids under nitrogen starvation conditions in wild-type Synechococcus, the ΔglgC strain, and the ΔglgC/ΔcrtG (2,2′-β-hydroxylase) strain. Levels of total carotenoids were measured as μg/OD by extracting pigments from Synechococcus cells with dimethylformamid (DMP, centrifugation in a microcentrifuge at 4° C. for 10 min then using the supernatant to quantify carotenoid content photometrically. The concentration of total colored carotenoids ([Carotenoid]) as μg/ml in the DMF extract was determined by the formula [Carotenoid]_{(μg/ml)}=(A₄₆₁−0.046×OD₆₆₄)×4. The deletion of the crtG gene was accomplished as described in Example 1. FIG. 5 shows an increase in carotenoid content during nitrogen deprivation relative to the nutrient replete conditions for all tested genotypes of Synechococcus. The increase was greatest in the ΔglgC/ΔcrtG strain. Dry weights of the strains were measured by weighing lyophilized cell pellets using a Mettler Toledo scale (Model XP26). Prior to measurement cell pellets were lyophilized under vacuum overnight.

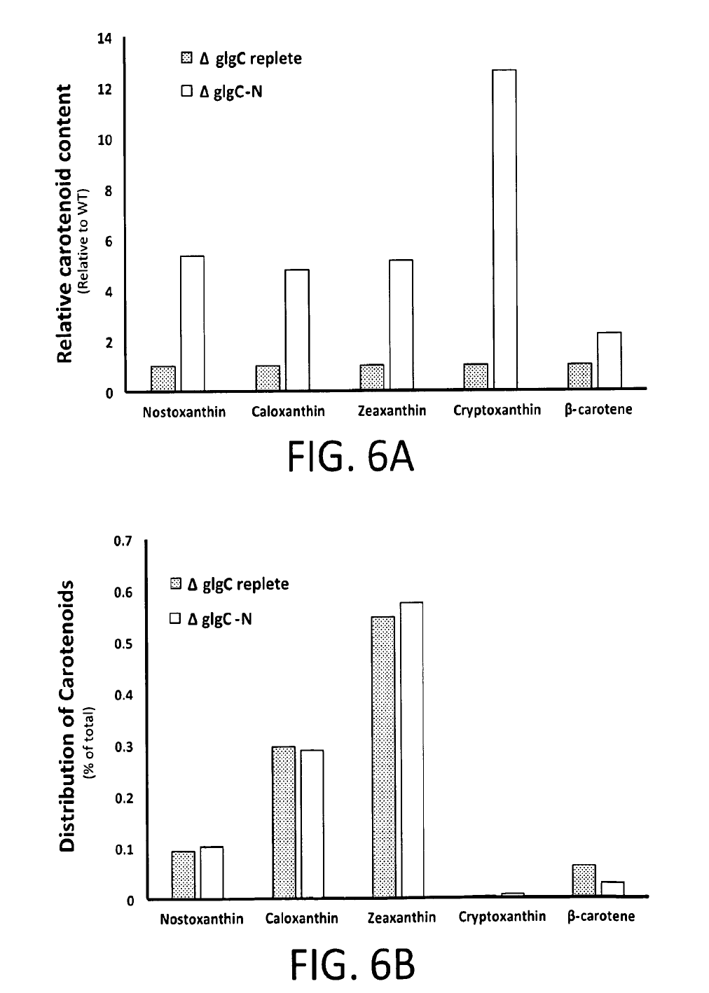

FIG. 6A is a bar graph showing the relative distribution of carotenoids for ΔglgC under both replete nutrient conditions and nitrogen starvation relative to wild-type Synechococcus under replete conditions. Specific carotenoid concentrations were determined by liquid chromatography-mass spectrometry (LC-MS) as described in Example 1, following a Bligh and Dyer extraction. For the Bligh and Dyer extraction, a sample containing 1 ml of cell culture (various cell densities) was added to 3.75 ml of a 1:1 chloroform/methanol and vortexed for 1 minute. Following the vortexing, 1.25 ml of chloroform was added and vortexed a second time for 1 minute. Subsequently 1.25 ml of 1 M NaCl was added and vortexed for another minute then centrifuged. After centrifugation the upper phase was discarded and the lower phase was collected through the protein disk with a Pasteur pipett. This samples was dried in a speed vac drying system, and resuspended in 200 μL 1:1 chloroform:methanol for analysis on the HPLC. Under nitrogen replete conditions the carotenoid content of ΔglgC was essentially the same as the wild-type Synechococcus (as shown by bar heights of around 1). Under nitrogen starvation of the ΔglgC strain, cryptoxanthin content increases about 13-fold; β-carotene content increased about 2-fold; and nostoxanthin, caloxanthin, and zeaxanthin each increased about 5-fold.

FIG. 6B was generated from the same data as FIG. 6A and shows the change in carotenoid distribution that resulted from nitrogen deprivation. Zeaxanthin increased slightly to around 57% of total carotenoids while β-carotene decreased from around 6% to around 3%. The relative amounts of nostoxanthin, caloxanthin, and cryptoxanthin remained essentially unchanged.

### Example 3—Nutrient Stress

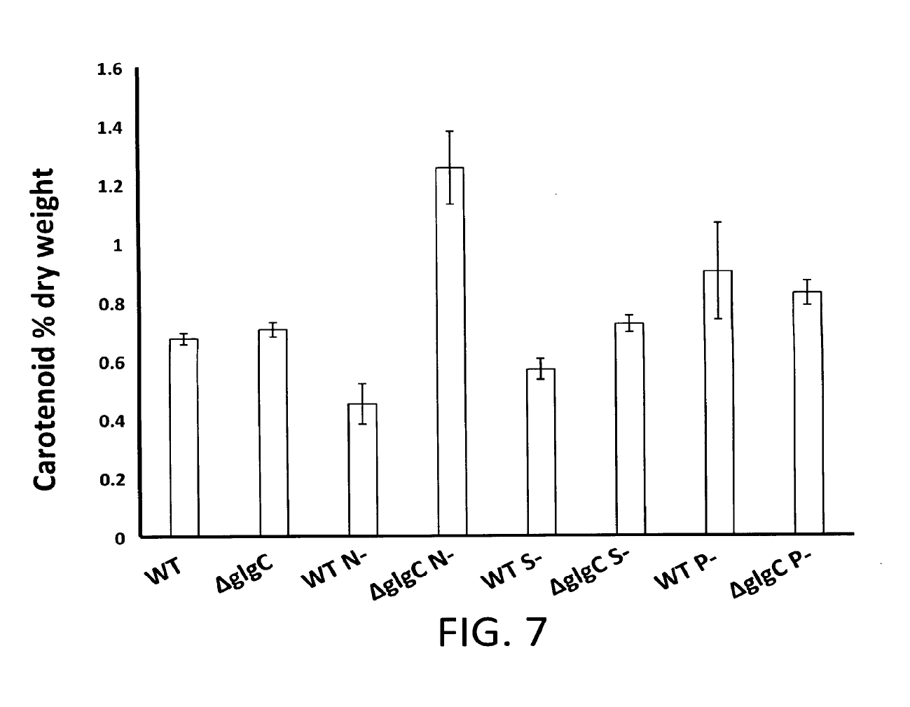

Both wild-type Synechococcus and ΔglgC were subjected to various types of nutrient starvation. Nutrient starvation was induced as described above, by washing the strain of interest with the nutrient free media then incubating that culture in the nutrient free media of interest for 48 hr. The incubation was done under standard growth conditions (BG-11 media under an atmosphere of 1% CO₂ and 100 μmoles photons m⁻² s⁻¹) for nutrient replete conditions. Sulfur deprivation was achieved by omitting MgSO₄ from BG-11 media with the other growth conditions the same. Phosphorous deprivation was achieved by omitting K₂HPO₄ from BG-11 media with the other growth conditions the same. Carotenoid content was measured 48 hours after transferring the cells to a nutrient deficient media. FIG. 7 shows the percent of total dry weight accounted for by carotenoids for both wild-type strains (denoted as WT) and ΔglgC mutants under nutrient replete, nitrogen deprivation, sulfur deprivation, and phosphorus deprivation. Carotenoid dry weight percentages were measured using the techniques described in Example 2. Wild-type Synechococcus cultured under replete conditions has approximately 0.7% carotenoids by dry weight. Wild-type Synechococcus cultured under nitrogen starvation conditions had a reduced level of carotenoids—only about 0.4%. Interestingly the highest percentage of carotenoids, 1.3%, was found in Synechococcus modified by deletion of glgC that was cultured under nitrogen-starvation conditions.

### Example 4—Enhancing the Carotenoid Biosynthesis Pathway

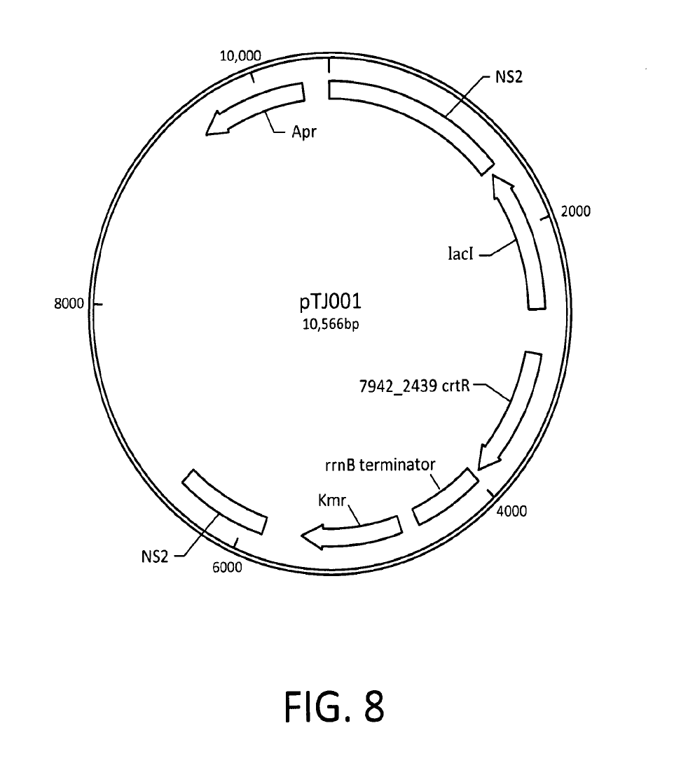

Zeaxanthin content may be increased by increasing the expression of the carotenoid hydroxylase crtR. crtR is a carotenoid 3,3′-hydroxylase that oxidizes β-carotene to zeaxanthin as shown in FIG. 1. To increase crtR expression in S. elongatus, the endogenous crtR gene from S. elongatus PCC 7942 (SEQ ID NO: 21) was transformed in the wild-type strain under control of the pTrc (IPTG inducible) promoter. FIG. 8 shows the vector used for the transformation which was a pTJ001/pMX570 plasmid backbone with addition of pTrc-crtR and a gene that conferred resistance to the antibiotics spectinomycin and streptomycin (SEQ ID NO: 23). The resulting strain was grown in standard conditions (BG-11 media under an atmosphere of 1% CO₂ and 100 μmoles photons m⁻²s⁻¹), and the crtR gene introduced by the vector was overexpressed through incubation with 1 mM IPTG. The overexpression of the crtR gene does not result in any alterations in growth, bilin (biochrome) content, or chlorophyll content. However, it does result in a significant increase in zeaxanthin. This increase in zeaxanthin is accompanied by a decrease in β-carotene.

### Example 5—Addition of Exogenous Carotenoid Biosynthesis Genes

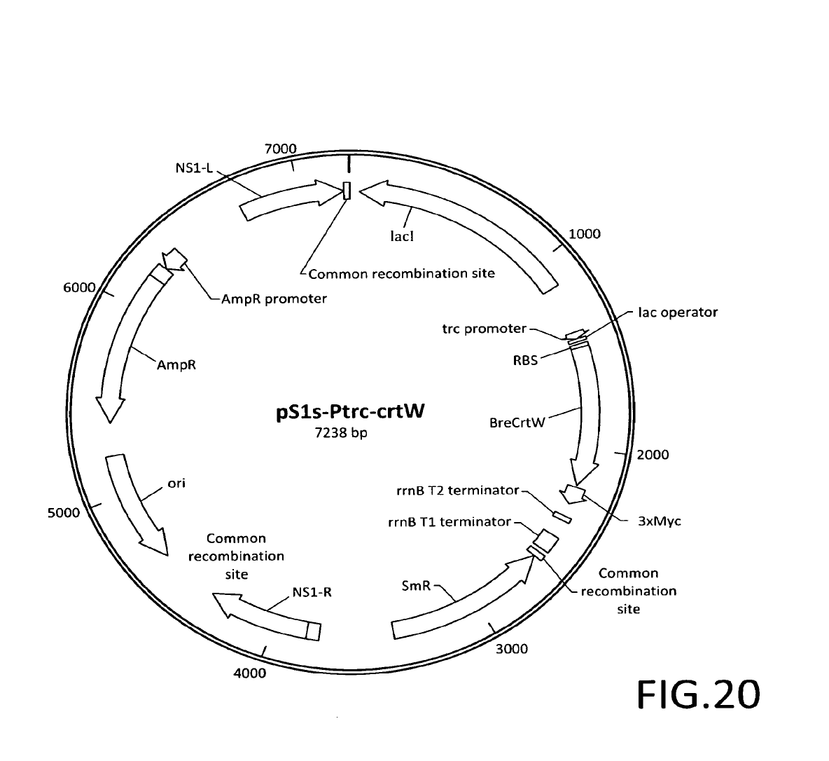

To produce canthaxanthin and astaxanthin in S. elongatus, the crtW gene from Brevindomonas sp. SD212 (SEQ ID NO: 29) under control of the pTrc (IPTG inducible) promoter was transformed in the ΔglgC mutant. The plasmid (pS1s-pTrc-crtW) that contains pTrc-crtW also contains a gene that confers resistance to the antibiotics spectinomycin and streptomycin. The pS1s-pTrc-crtW plasmid has SEQ ID NO: 30 and is shown in FIG. 20. Transformants that integrated this vector into their genome were selected by their resistance to spectinomycin and streptomycin.

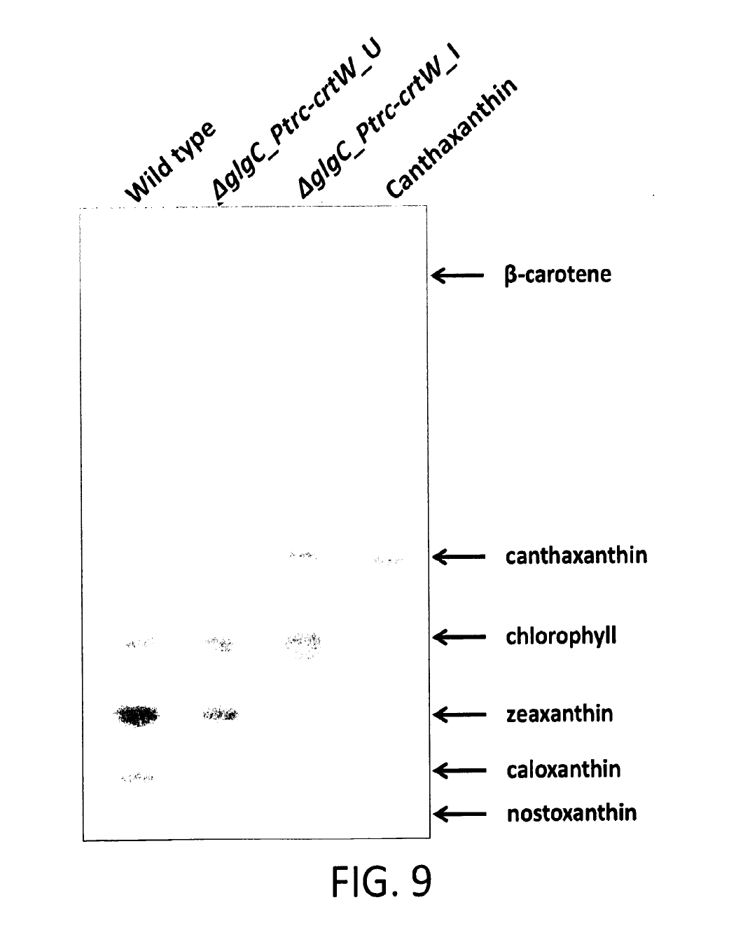

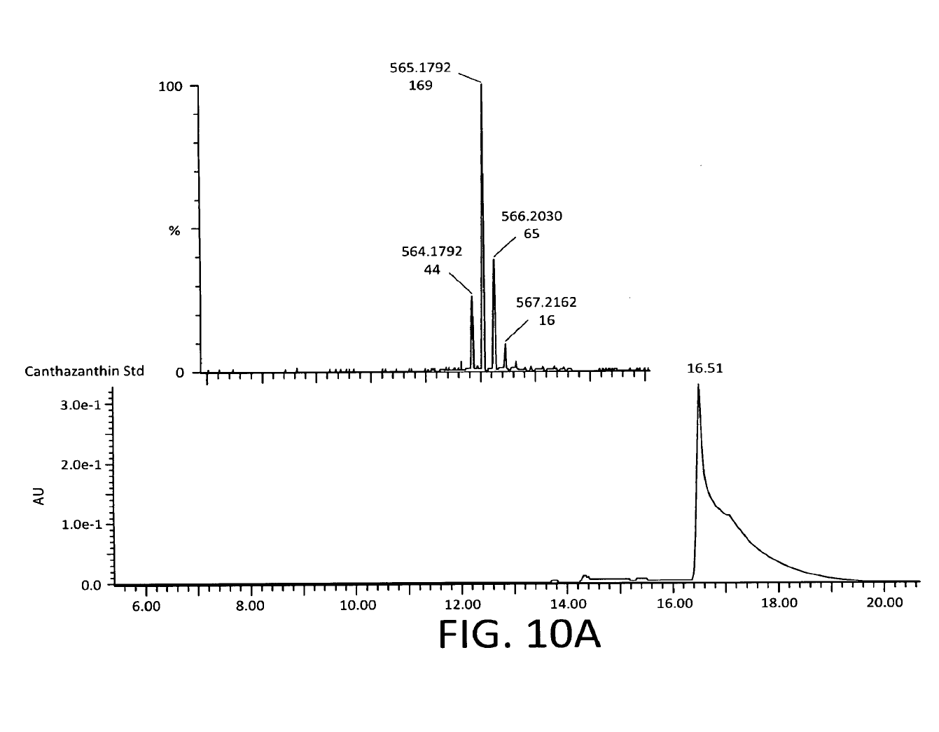

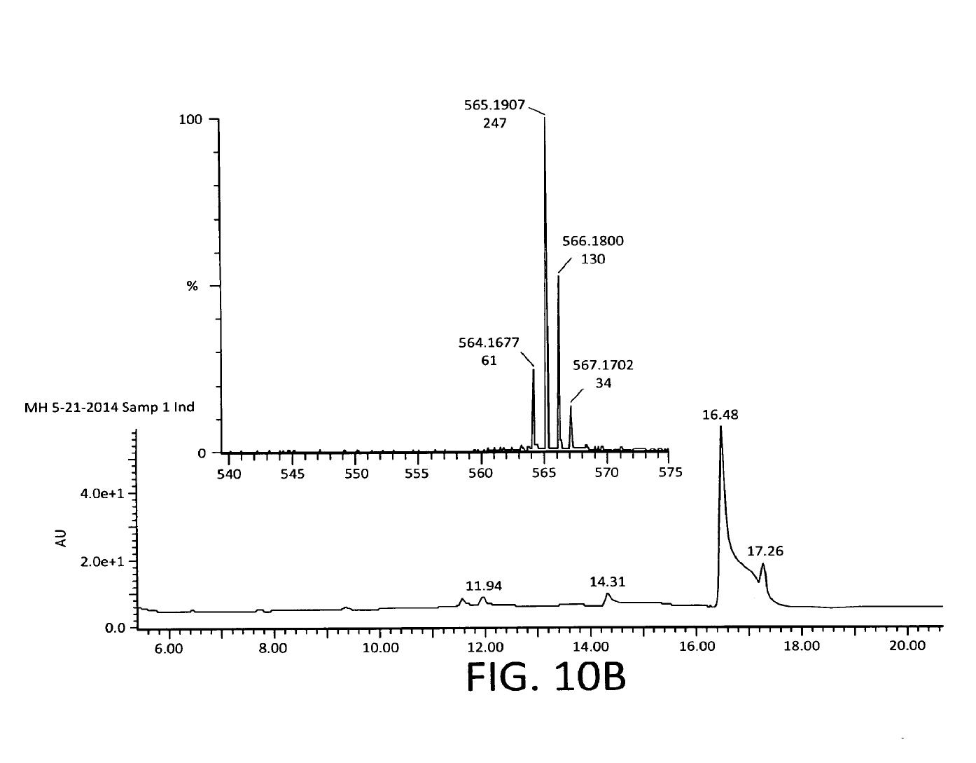

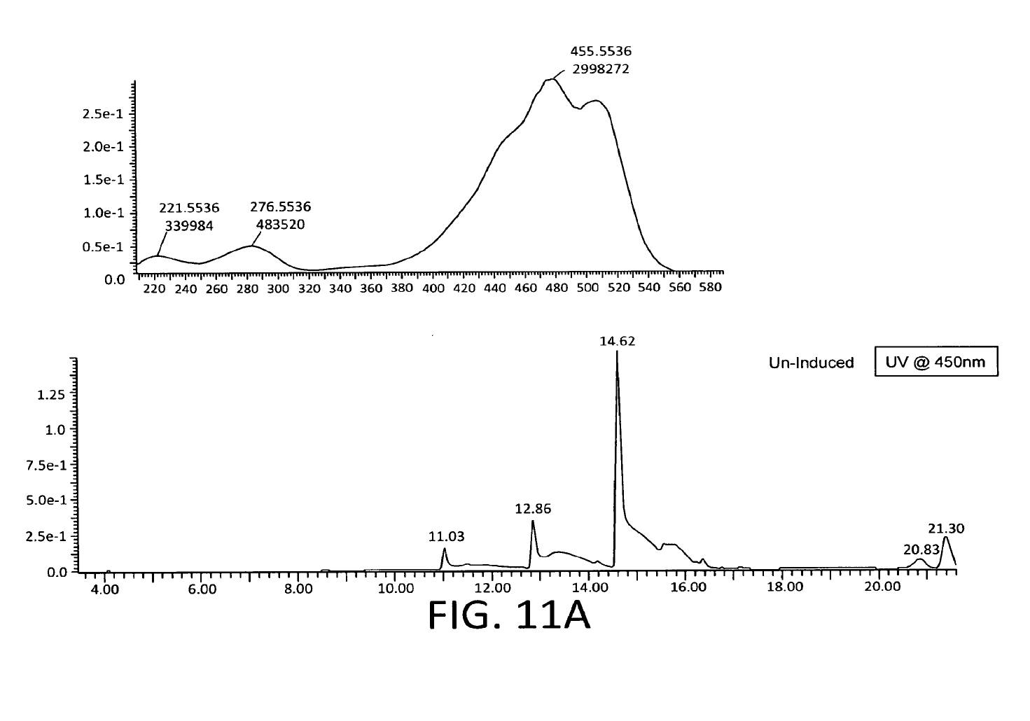

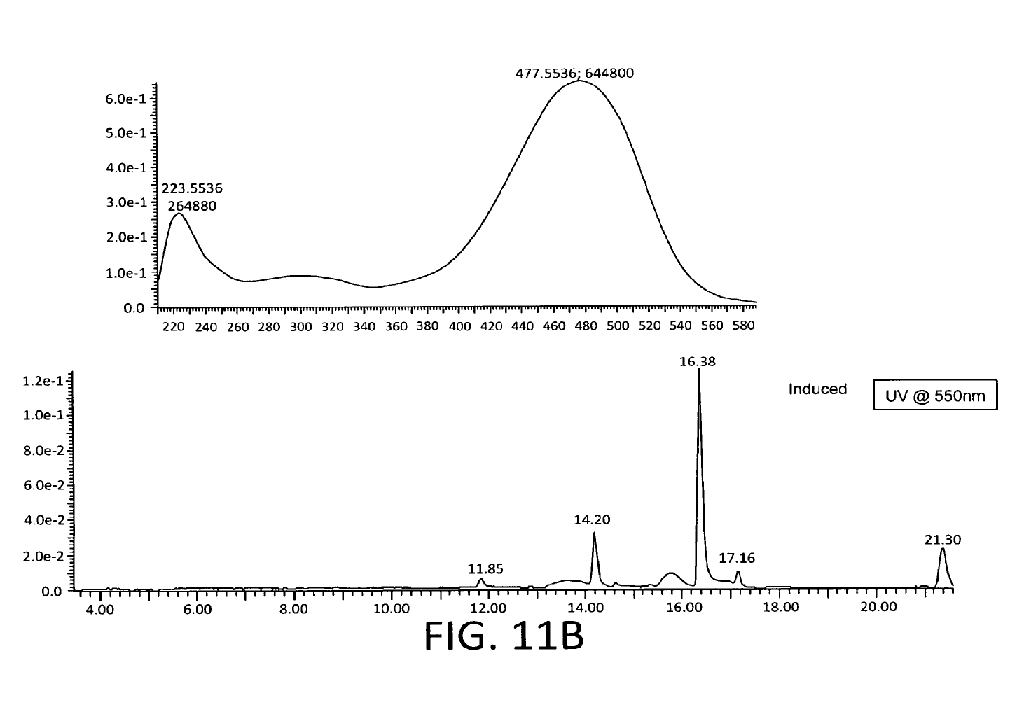

When the pTrc-crtW/ΔglgC mutant is grown in BG-11 media it produces a small amount of red carotenoid that can be visualized on a silica TLC plate. Cells with an optical density of 1 at 750 nm were spun down at 10,000×g. Pigments were extracted with acetone. Following an evaporation of the acetone, samples were loaded on silica TLC plate. FIG. 9 shows the comparison of pTrc-crtW/ΔglgC mutant in the presences of IPTG to induce crtW, pTrc-crtW/ΔglgC mutant without IPTG, and a canthaxanthin standard (Fisher Scientific). The red carotenoid from the mutant has the same Rf value on a TLC plate as the canthaxanthin standard. Additionally, FIG. 10 shows that when the cultures of pTrc-crtW/ΔglgC are analyzed by LC-MS the red carotenoid exhibits the same retention time and molecular weight as the canthaxanthin standard. This indicates that the carotenoid biosynthesis pathway shown in FIG. 1 in the pTrc-crtW/ΔglgC mutant has been successfully modified to create canthaxanthin from β-carotene rather than zeaxanthin. Induction of crtW greatly increased synthesis of canthaxanthin as shown by the difference between the uninduced and induced sample lanes in FIG. 9 and by the differences between the top and bottom chromatograms of FIG. 11. The samples used for both the top and bottom chromatograms shown in FIG. 11 are Synechococcus with glgC deleted and addition of exogenous crtW under the control of an IPTG inducible promoter. Culture conditions were the same for the two samples. The difference is that no IPTG was added for the sample shown in the top chromatogram. This lowered IPTG likely causes less expression of crtW. However, the bottom chromatogram shown in FIG. 11 shows a sample that was incubated with 1 mM IPTG for 24 h to induce expression of crtW. As shown by the shift in the major peak from the top chromatogram (uninduced) to the bottom chromatogram (induced) expression of crtW changes the carotenoid synthesis pathway from primarily synthesizing zeaxanthin to producing canthaxanthin. As the maximum absorbance of zeaxanthin (the major species in the absence of IPTG) is at 450 nm, this is the wavelength used for analyzing the elution profile of the uninduced strain. As the maximum absorbance of canthaxanthin (the major species in the presence of IPTG) is at 550 nm, this is the wavelength used for analyzing the elution profile of the induced strain.

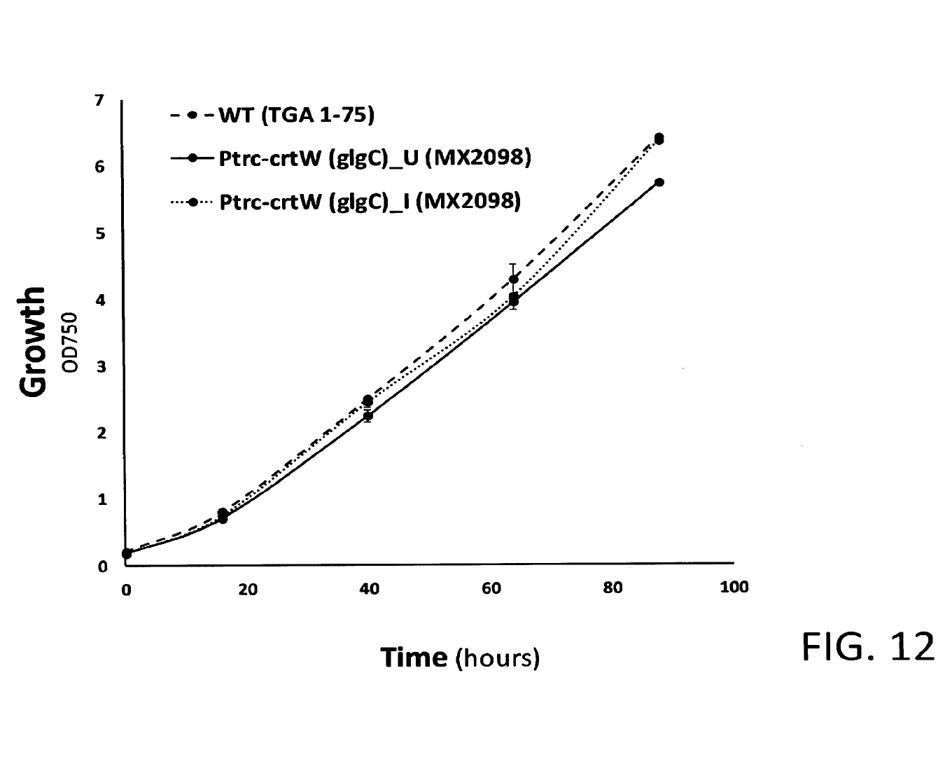

After 24 h of incubation with 1 mM IPTG to induce expression of crtW: β-carotene, zeaxanthin, caloxanthin, and nostoxanthin are greatly reduced as determined by both TLC and LC-MS in pTrc-crtW/ΔglgC (FIGS. 9-11). In spite of this change to the carotenoid biosynthesis pathway, the S. elongatus continued to grow under standard conditions (BG-11 media, 100 μmoles photons m⁻² s⁻¹ and an atmosphere of 1% CO₂). As shown in FIG. 12, the growth of pTrc-crtW/ΔglgC measured as optical density at 750 nm is not affected to a significant degree by incubation with IPTG.

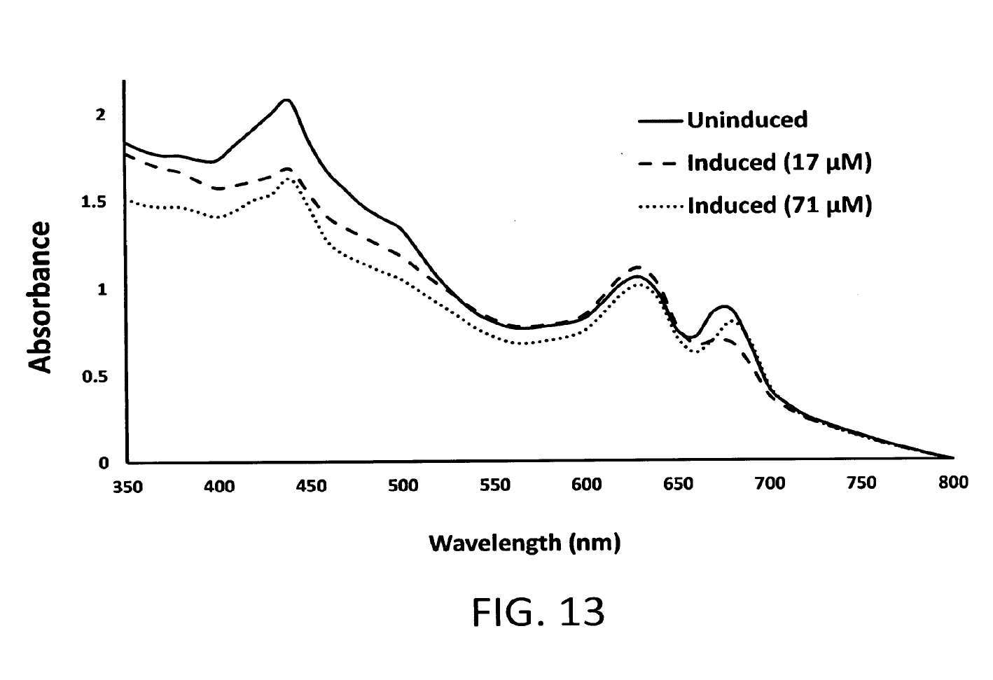

FIG. 13 confirms that addition of IPTG induces crtW expression in the pTrc-crtW/ΔglgC strain. Since CrtW is an iron dioxygenase, overexpression is expected to reduce iron in the media. Loss of chlorophyll in pTrc-crtW/ΔglgC upon induction of crtW with IPTG was attributed to this iron deficiency. As shown in FIG. 13, addition of 1 mM IPTG to induce crtW expression lead to a decrease in absorbance at 680 nm (corresponding to chlorophyll a) as compared to the uninduced pTrc-crtW/ΔglgC mutant grown in standard conditions. Significantly, this decrease in absorbance at 680 nm was counteracted by the addition of 54 mM ferric chloride to bring the final iron concentration in the media from 17 mM to 71 mM. The normal amount iron in BG-11 is only 17 mM so the high iron level of 71 mM represents a fourfold increase of ferric chloride in the media.

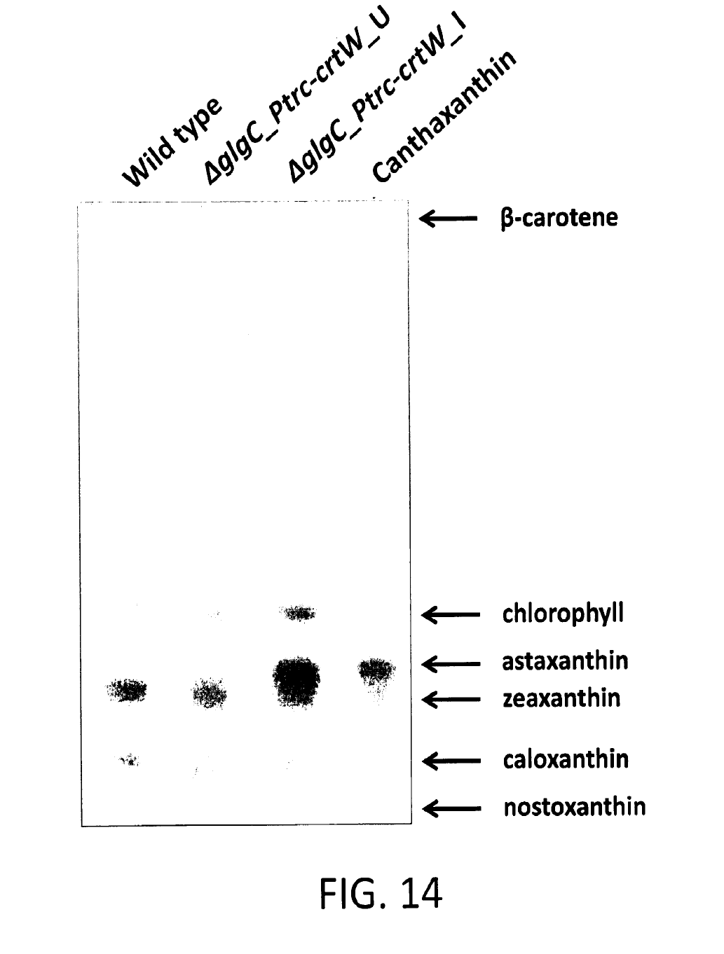

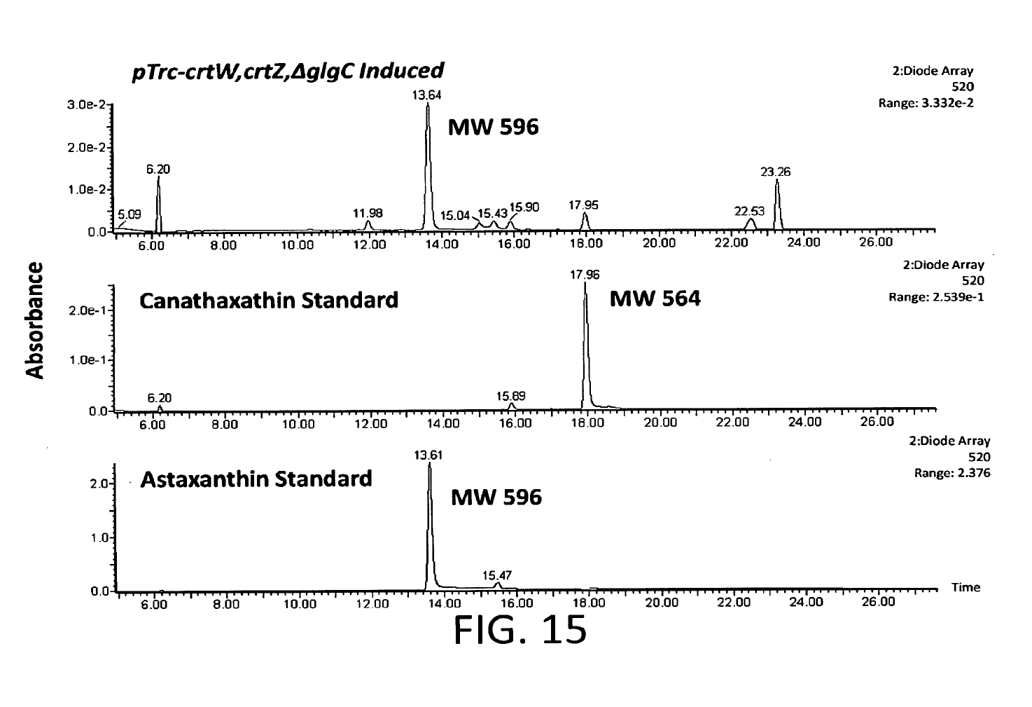

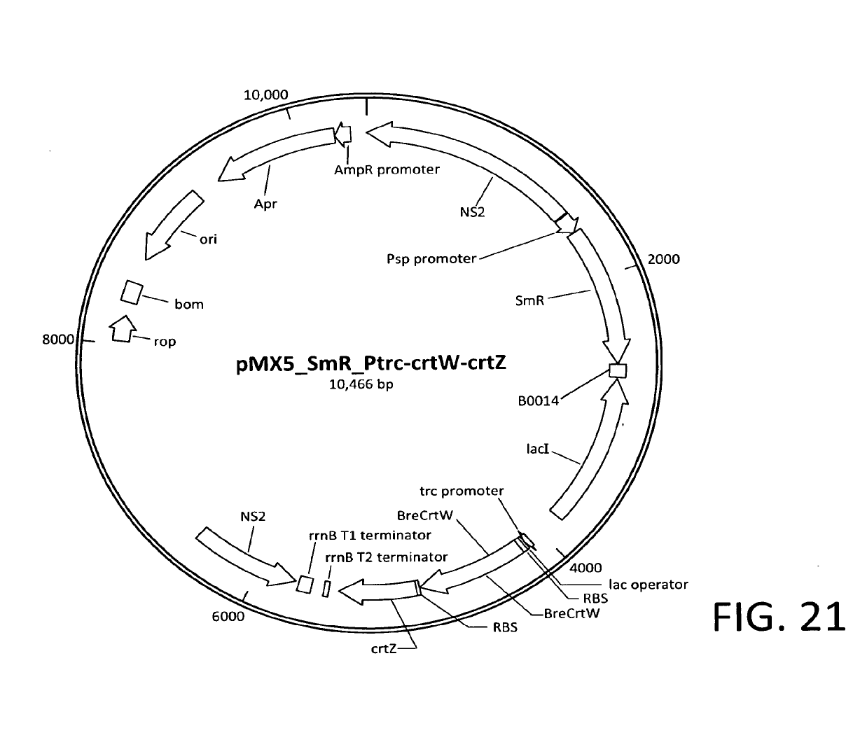

FIG. 14 shows synthesis of astaxanthin, a carotenoid not naturally produced by Synechococcus, produced by the pTRC-crtW-crtZ/ΔglgC strain under induction with IPTG (created by transforming the ΔglgC strain with the plasmid shown in FIG. 21). The TLC gel shown in FIG. 14 was prepared using a similar procedure as the TLC gel shown in FIG. 2. pTrc-crtW-crtZ/ΔglgC with crtW and crtZ induced is compared to an astaxanthin standard (Fisher Scientific) in the far right lane. In pTrc-crtW-crtZ/ΔglgC that is incubated with 1 mM IPTG, β-carotene, zeaxanthin, caloxanthin, and nostoxanthin are greatly decreased and are replaced mainly with astaxanthin as shown on FIG. 14. There are additional carotenoids that are produced, most of notably canthaxanthin; however, these carotenoids are a small fraction of the total and not shown on FIG. 14. In addition, FIG. 15 shows a LC-MS chromatogram of the red carotenoids run in the ΔglgC pTrc-crtW-crtZ lane of the gel shown in FIG. 14. The chromatogram includes a large peak at 13.64 min that has the same retention time and molecular mass as an astaxanthin standard (FIG. 15, masses are determined as described above). There is also a much smaller peak that has the same retention time and mass as canthaxanthin.

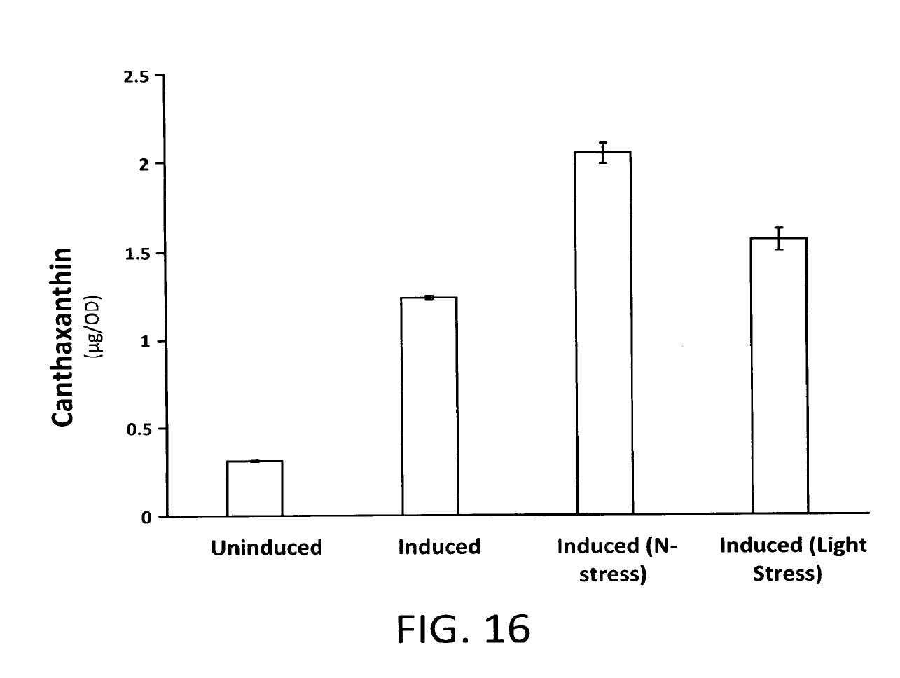

FIG. 16 shows an increase in canthaxanthin production in pTrc-crtW/ΔglgC when crtW is induced and a further increase when subjected to nitrogen stress (this strain was produced by transforming the ΔglgC strain with the plasmid shown in FIG. 20). Cells of the pTrc-crtW/ΔglgC strain grown under standard conditions without inducing crtW resulted in 0.31 μg/OD of cantaxanthin. Addition of 1 mM IPTG to induce crtW lead to an increase in canthaxanthin to about 1.24 μg/OD. The pTRC-crtW/ΔglgC strain was grown to a concentration of about 6×10⁶ cells/mL as determined by optical density at 750 nm then nitrogen stressed by placing the cells in placed in nitrogen free media as described above. Induction by addition of 1 mM IPTG was performed at the same time that nitrogen deprivation was initiated. This combination let to a further increase in canthaxanthin production to 2.04 μg/OD—approximately a 65% increase over crtW induction in nitrogen replete conditions. Induction of crtW combined with light stress (culturing with light levels at 500 μmoles photons m⁻² s⁻¹) increased canthaxanin production to 1.56 μg/OD—approximately a 27% increase over crtW induction in nitrogen replete conditions. Canthaxanthin concentrations were determined by LC-MS in comparison to a canthaxanthin standard (Fisher scientific).

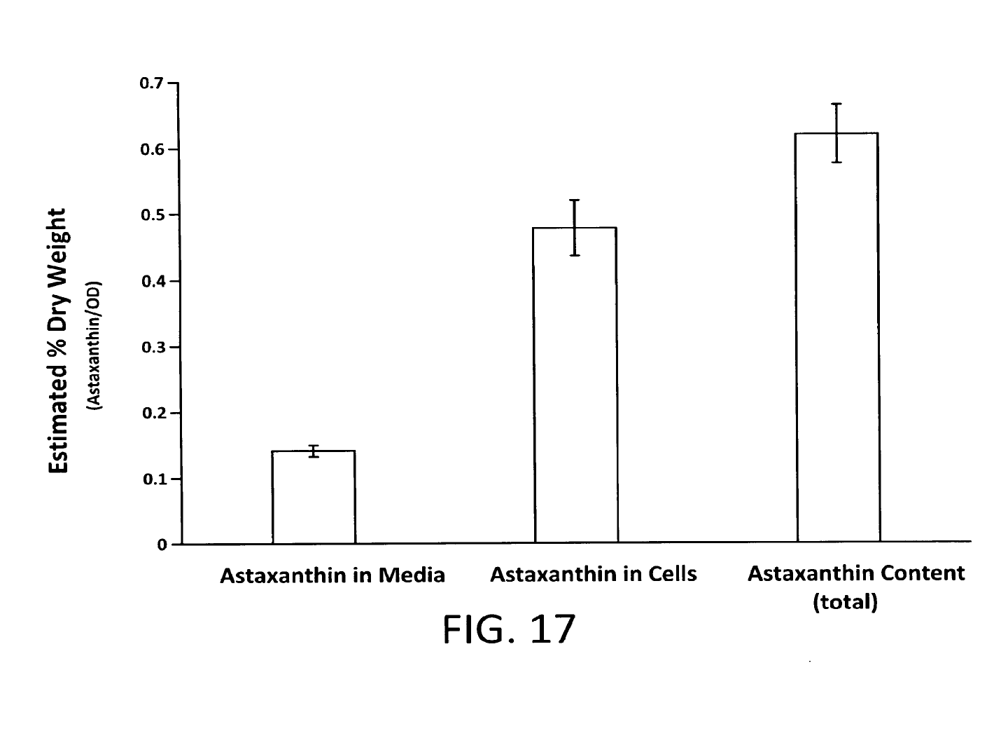

FIG. 17 shows production of astaxanthin in pTrc-crtW-crtZ/ΔglgC when crtW and crtZ are induced. Cells of the pTrc-crtW-crtZ/ΔglgC strain grown under standard conditions without inducing crtW or crtZ resulted in no detectable astaxanthin. Addition of 1 mM IPTG to induce crtW and crtZ lead to production of astaxanthin inside the cells at a level of 1.67 μg/OD after 5 days. Unlike the canthaxanthin producing strains, the astaxanthin producing strains secrete a significant amount of the product into the culture media. The astaxanthin in the culture media turns the media pink. On a per cell basis, the astaxanthin in the media is 0.5 μg/OD. When these two values are added together they give a final astaxanthin concentration of 2.16 μg/OD. A value that is approximately equal to the amount of canthaxanthin produced by pTRC-crtW/ΔglgC under conditions of nitrogen stress as shown by the third column of FIG. 16.

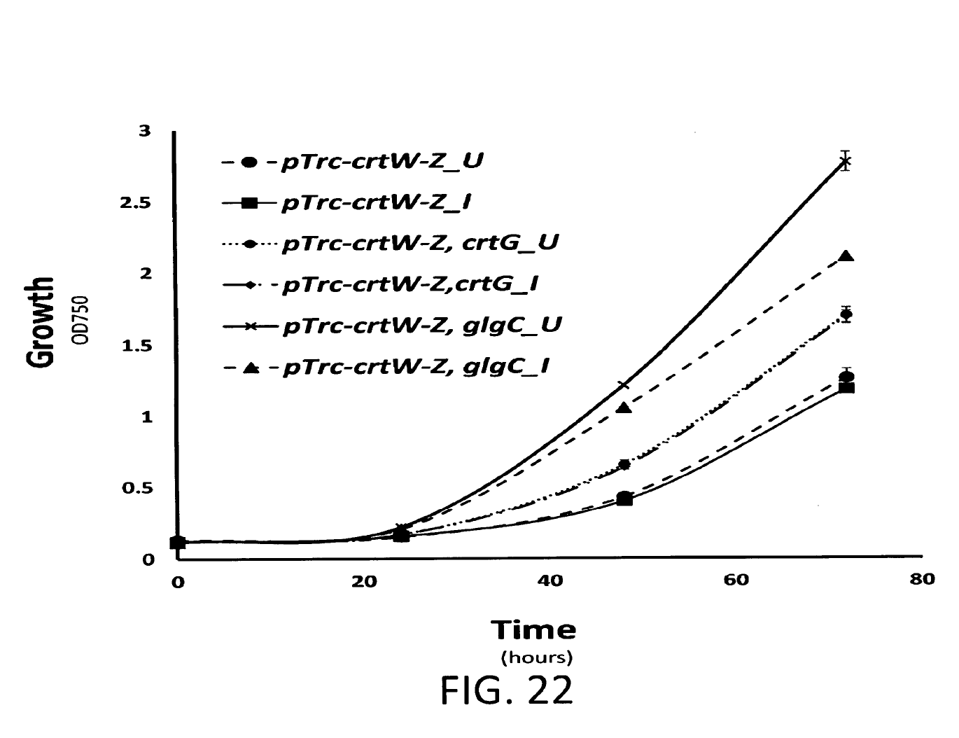

FIG. 22 is a growth curve comparing growth as measured as optical density at 750 nm of six different strains of Synechococcus over a 75 day period. All of the six strains were modified by addition of crtW under control of the pTrc promoter and addition of crtZ. The light-blue line corresponds to a strain that did not have crtW induced. The orange line corresponds to a strain that has the crtW induced by addition of IPTG. Both of these strains exhibited the lowest levels of growth after 75 days. The gray and yellow lines (gray line is mostly covered by the yellow line) correspond to a strain that additional has deletion of the crtG gene. The gray line represents growth when crtW is uninduced and the yellow line represents growth when crtW is induced. Between these two strains the presence or absence of crtW did not affect growth. Deletion of crtG increased growth after 75 days as compared to the strains which correspond to the light-blue and orange lines. The green line corresponds to a strain that has the glgC gene deleted and crtW uninduced. Deletion of glgC even without crtW further increases growth after 75 days. The most rapid growth as shown by the blue line was observed in a strain that has glgC deleted and crtW induced. Thus, the combination of addition of crtW and crtZ with deletion of glgC results in much stronger growth than any of the modifications individually.

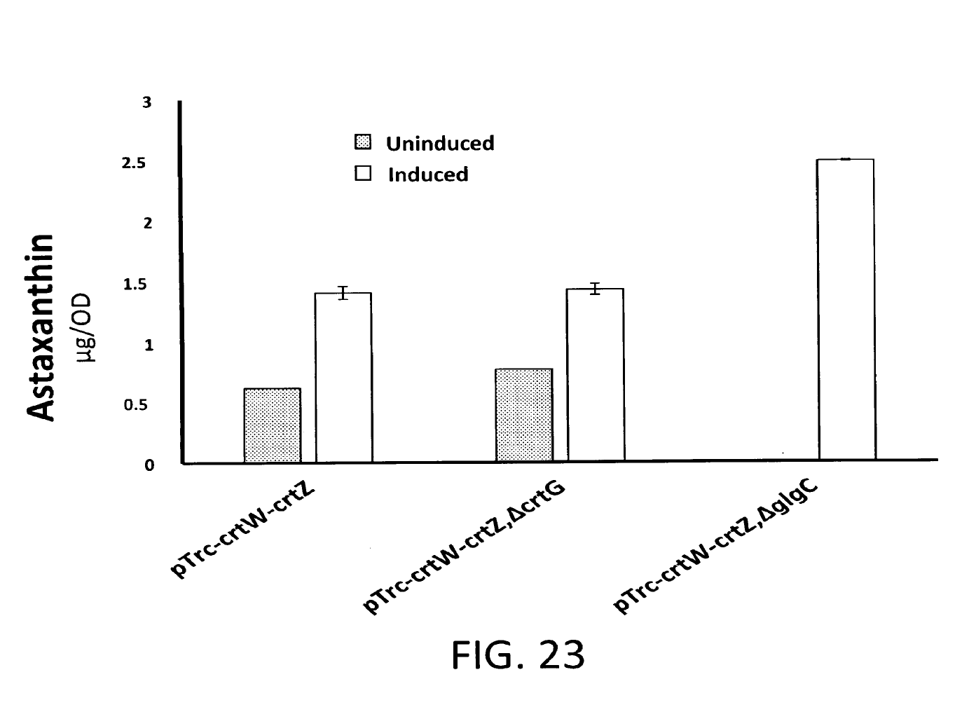

FIG. 23 shows astaxanthin levels in the six strains discussed in FIG. 22. The astaxanthin levels were measured as μg/OD by the same technique used to generate the data for FIG. 5. In FIG. 23 the orange bars represent strains for which crtW and crtZ were not induced and the blue bars represent strains for which the crtW and crtZ were induced. In all strains induction of crtW increased astaxanthin yields. The pTrc-crtW-crtZ and pTrc-crtW-crtZ/ΔcrtG strains produced roughly the same amounts of astaxanthin. But the pTrc-crtW-crtZ/ΔglgC strain produced no astaxanthin when crtW as not induced and produced the highest levels of astaxanthin, around 2.5 μg/OD, when crtW was induced. Thus, the deletion of glgC significantly increases the amount of astaxanthin produced as between two strains that are both modified with the addition of crtW and crtZ.

### Example 6—Light Stress

FIG. 16 also shows carotenoids quantitifed from the pTrc-crtW/ΔglgC strain after incubation under high light (500 μmol photon m-2 s-1) for six days. As can be seen, high light stress increases carotenoid content from 1.23 μg/OD to 1.56 μg/OD an increase of (27%).

1. A method of producing a carotenoid, the method comprising:
culturing a modified Cyanobacterium, the modified Cyanobacterium having decreased glycogen production; and
subjecting the modified Cyanobacterium to a stress condition during culturing, wherein the modified Cyanobacterium when subjected to the stress condition produces the carotenoid at a level greater than the corresponding wild-type Cyanobacterium subject to the same stress condition.

2. (canceled)

3. The method of claim 1, wherein the carotenoid comprises zeaxanthin, astaxanthin, canthaxanthin, or a combination thereof.

4-6. (canceled)

7. The method of claim 1, wherein the modified Cyanobacterium is modified by one of (i) deletion or inactivation of a glycogen pathway gene or (ii) by addition of a regulatable, exogenous promoter that controls transcription of a glycogen pathway gene.

8. (canceled)

9. The method of claim 7, wherein the regulatable, exogenous promoter, when activated by a regulator, controls transcription of the glycogen pathway gene, the regulator comprises a nutrient, and the stress condition comprises deprivation of the nutrient.

10. The method of claim 7, wherein the glycogen pathway gene is glgC.

11. The method of claim 1, wherein the stress condition comprises at least one of high light, high salt, nutrient deprivation, nitrogen deprivation, sulfur deprivation, phosphorous deprivation, or iron deprivation.

12-14. (canceled)

15. The method of claim 1, wherein the modified Cyanobacterium further comprises deletion of a crtG gene.

16. The method of claim 15, wherein the carotenoid comprises zeaxanthin.

17. The method of claim 1, wherein the modified Cyanobacterium further comprises an exogenous carotenoid ketolase gene.

18-19. (canceled)

20. A modified Cyanobacterium comprising:
a deleted or inactivated glycogen pathway gene,
wherein the modified Cyanobacterium, when subjected to a stress condition, produces a carotenoid at a level greater than the corresponding wild-type Cyanobacterium subject to the same stress condition.

21-23. (canceled)

24. The modified Cyanobacterium of claim 20, further comprising an exogenous carotenoid ketolase gene.

25-26. (canceled)

27. The modified Cyanobacterium of claim 20, wherein the glycogen pathway gene is glgC.

28-31. (canceled)

32. A method for generating modified Cyanobacterium, the method comprising:
deleting or inhibiting a glycogen pathway gene or addition of a regulatable, exogenous promoter that controls transcription of the glycogen pathway gene; and
adding an exogenous polynucleotide encoding an enzyme involved in the carotenoid biosynthesis pathway;
wherein the modified Cyanobacterium produces a carotenoid at a level greater than the corresponding wild-type Cyanobacterium.

33-34. (canceled)

35. The method of claim 32, 33, or 311, wherein the glycogen pathway gene is glgC.

36. The method of claim 32, wherein the regulatable, exogenous promoter, when activated by a regulator, controls transcription of the one or more glycogen pathway genes and the regulator is a nutrient, and
wherein the absence of the nutrient causes the modified Cyanobacterium a stress condition.

37. The method of claim 32, wherein the enzyme involved in the carotenoid biosynthesis pathway is a carotenoid ketolase.

38-39. (canceled)

40. The method of claim 32, further comprising addition of an exogenous crtZ gene and wherein the carotenoid is astaxanthin.

41. The method of claim 32, wherein the enzyme involved in the carotenoid biosynthesis pathway is a β-carotene hydroxylase.

42. The method of claim 41, wherein the carotenoid is zeaxanthin.

43. The method of claim 32, wherein the modified Cyanobacterium when subjected to a stress condition produces the carotenoid at the level greater than the corresponding wild-type Cyanobacterium subjected to the same stress condition.

44-45. (canceled)
